SEQUENCE LISTING (31 sequences)

SEQ ID NO: 1 - DNA, 33 bp, Artificial Sequence
  ProB55F gene specific forward cloning primer
atcgagtcgaccatggagagcgcagaggagcct

SEQ ID NO: 2 - DNA, 32 bp, Artificial Sequence
  ProB55R gene specific reverse cloning primer
caagccatggcgcttcatccaccagatgatgg

SEQ ID NO: 3 - DNA, 22 bp, Artificial Sequence
  ProSeqF pDBLeu forward vector specific primer
gaataagtgcgacatcatcatc

SEQ ID NO: 4 - DNA, 22 bp, Artificial Sequence
  ProSeqR pDBLeu reverse vector specific primer
gtaaatttctggcaaggtagac

SEQ ID NO: 5 - DNA, 755 bp, Homo sapiens
  misc_feature 6, 22, 30, 42, 62, 454, 530, 536, 561, 632, 639, 640,642, 643, 645, 646, 647, 648, 649, 650, 651, 652, 653, 654, 655,656, 657, 659, 661, 662, 666, 676, 680, 705, 724, 754: n = A,T,C or G
aaattnaaaccttgaaaacccnacaatgtntgatgtatatanctatctattcgatgatgaanataccccaccaaacccaaaaaaagagggtgggtcgacccacgcgtccgcccacgcgtccgcccacgcgtccgctacagcacagatcagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcaagcgatgccagagagaatatccagagattctttggccatggtgcggaggactcgctggctgatcaggctgncaatgaatggggcaggagtggcaaaaaccccaatcactttccgacctgctggcctgcctgagaaatactgagcttnctcttnctctgttctcaagagatctggctgngaggccttaaggcagggatacaaagcggggagagggtacacaatgggtatctaataaatacttaagaaggnggggaannannannnnnnnnnnnnnanannaaangggcggccgntaantaaagtaaaacgtcaaacttttaantaaagtaaaccggccgccnccggggggggagcttttggaccttttttcnc

SEQ ID NO: 6 - DNA, 758 bp, Homo sapiens
  misc_feature 3, 4, 343, 536, 563, 575, 608, 611, 619, 662, 692, 695,711, 727, 733, 754, 756, 757: n = A,T,C or G
ttnnatttaacccttgtaaataccctacaatggatgatgtatataactatctattcgatgatgaagataccccaccaaacccaaaaaaagagggtgggtcgacccacgcgtccggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcngcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcattccagggggatataatgctttaaggcgcatgtacacagatattcangaaccaatgctgagtgctgcacaagancagtttggtggnaatccattttgctttcttggtgagcaatacatnctntggtgaangtagtcaaccttccgtacagaaaatagagatcactacccaatncatgggcttccacagactttccagaagttnatnaagctttcaaccgggnacttgccagcacttgngggggnggccttctgggtagtacttgncnng

SEQ ID NO: 7 - DNA, 764 bp, Homo sapiens
  misc_feature 4, 5, 10, 555, 563, 568, 622, 624, 631, 633, 634, 635,636, 637, 638, 639, 640, 641, 642, 643, 644, 645, 646, 647, 648,649, 650, 651, 652, 653, 654, 655, 656, 657, 658, 659, 660,661, 663, 671, 672, 673, 696, 699, 706, 707, 716, 718: n = A,T,C or G
  misc_feature 731, 735: n = A,T,C or G
tttnnatccntaaaaccttggaaaaccctacaatggatgatgtatataactatctattcgatgatgaagataccccaccaaacccaaaaaaagagggtgggtcgacccacgcgtccggcagctcagctacagcacagatcagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgangccctcanggcanggatacaaagcggggagagggtacacaatgggtatctaataaatacttaaagangngggaaanannnnnnnnnnnnnnnnnnnnnnnnnnnnnanaaaaaaannngggggcgggccgttaagtaaagnaanaacgttnnacttttaantnaagtaaccgggcngccnccgggggggggagctttgggacttttttc

SEQ ID NO: 8 - DNA, 754 bp, Homo sapiens
  misc_feature 2, 3, 4, 7, 10, 15, 29, 493, 534, 628, 630, 645, 646,648, 649, 650, 651, 652, 653, 654, 655, 656, 657, 658, 659, 660,661, 662, 663, 664, 665, 666, 667, 668, 669, 670, 671, 672,673, 674, 675, 676, 677, 679, 680, 681, 682, 684, 685: n = A,T,C or G
  misc_feature 686, 687, 688, 689, 690, 691, 692, 693, 694, 695, 696,697, 698, 699, 700, 701, 702, 703, 705, 706, 710, 714, 715, 716,717, 723, 734, 739, 740, 748: n = A,T,C or G
tnnnatnagnatacnctttgaaaaaccangacaatggatgatgtatataactatctattcgatgatgaagataccccaccaaacccaaaaaaagagggtgggtcgacccacgcgtccgcccacgcgtccgcagctacagcacagatcagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaaagaccccaatcacttncgacctgctggcctggctgagaaatactgagctttctcttnctctgctctcaagagatctggctgtgaggccctcagggcagggatcaaaagcggggagagggtacacaatgggtatctaataaatacttaagangngggaaaaaaaaaaannannnnnnnnnnnnnnnnnnnnnnnnnnnnnntnnnntnnnnnnnnnnnnnnnnnnnnannaaanaaannnngggggnggccctttaanaaaannaaaaacgncaacct

SEQ ID NO: 9 - DNA, 776 bp, Homo sapiens
  misc_feature 19, 26, 36, 44, 69, 84, 87, 93, 164, 168, 183, 202, 209,227, 234, 245, 252, 257, 260, 306, 316, 323, 362, 365, 372,387, 395, 396, 399, 403, 417, 425, 444, 457, 474, 486, 501,511, 515, 528, 532, 537, 544, 552, 578, 583, 589, 619: n = A,T,C or G
  misc_feature 626, 634, 645, 648, 659, 663, 695, 697, 737, 744, 758,765, 766, 767, 769, 770, 775: n = A,T,C or G
tccgaaaacccttttgaanacgcccnagagacattnaccaaacntctggctgatagaagtccaaagctncaccgcggtggcggncgntacttntttagagctcgacgtcttacttacttagcggccgccctttattttttttttttttttttttttttttcccncctnttaagtatttattanatacccattgggtaccctntccccgntttgtatccctgccctgngggcctnacagccagatntcctganagcanagngaaaaggaagctcattttttttcaggcaggccaccaggtcggaagngattggggtntttgccnctcctgccccattcattggcaagcctgatcagccagggngtnctccccnccatggccaaaaaanttttggannttntttntggcatcggtgatnacttttgnagcccaggcccccccaggnccccttttgggancattatagttcccccgngcatggaaagtntttgctgaccgatgnaattggcttntttnattgtaaaaaaangctnttcanatgtccnagcccatnaaaagctttgccaaaggaacaaaaanaaacntttggntggtgaccccccagggacccaaggggcaanaaaaccnggccccgngaaaaagcttnatnggggcttgaantggngctggaaccgggaaccccttgggccggccccntngggccggccccttggggtcaaccccccccttttttttttnggggttnggggggggggattnttcatannncnnaaaana

SEQ ID NO: 10 - DNA, 896 bp, Homo sapiens
  misc_feature 5, 545, 584, 594, 598, 616, 619, 621, 631, 644, 654,665, 677, 697, 703, 713, 714, 749, 753, 771, 772, 797, 799, 800,830, 833, 841, 844, 856, 859, 865, 879, 891, 893, 895: n = A,T,C or G
agccncaaccttgattggagacttgaccaaacctctggcgaagaagtccaaagctccaccgcggtggcggccgttacttacttagagctcgacgtcttacttacttagcggccgccctttttttttttttttgcttctttttaatgcttttattctacataaattactaccataggctaatgtttaaaaagcaaataaactggacagatgcaggacaaaatctggtcacccaactataaaaggtgatgtttttaaaaaattacaataaatgcagaagtgatgcatgcagtagccttaattcccactgttccagaaaagaaaaatacagaaaaacccacacatcttactgtactccaccttaaaatgcatcatattgggtttgtttataacagcacagaattccaagagtcaaaatgaaataaagcaggtattttaaagtttaagagccgttatcaaaaataaattacattttttcaagatacagaaatgctgctatgatggctgggagcccagtaaccttttcaatagctgcattgatatcactnctggttgctattagagcttgcaaggttgctttcaccggntcaaaaaatnccantggcacttgagttggttncangntggtggctgnaaatctggacttnttgggatttntggaggctggngggatttacctncaaccaaggaaccctggaanccattntggctggaannaacctggctggatgttccaaggttcaagggggttnccgngtgggggggaccttgggnntttcactaagggggggggggttagnannccatttagtttccccgaaaaagcctccaanggntttctaanggncccccaaaccanganataaancctgggatgaaggncccgggctttcntntnt

SEQ ID NO: 11 - DNA, 907 bp, Homo sapiens
  misc_feature 3, 173, 176, 180, 187, 195, 197, 198, 204, 209, 214,221, 239, 246, 248, 254, 257, 264, 269, 272, 282, 287, 289, 293,295, 306, 322, 371, 383, 395, 401, 407, 410, 446, 469, 494,500, 509, 511, 525, 526, 537, 568, 588, 591, 596, 599: n = A,T,C or G
  misc_feature 607, 615, 622, 626, 650, 657, 661, 670, 672, 683, 691,697, 709, 710, 718, 736, 743, 762, 773, 775, 782, 791, 796, 797,824, 829, 840, 850, 854, 856, 861, 865, 868, 869, 877, 889,898, 899, 904: n = A,T,C or G
ttnaatcaagccgacaaccttgattggagacttgaccaaacctctggcgaagaagtccaaagctccaccgcggtggcggccgttacttacttagagctcgacgtcttacttacttagcggccgcccttttttttttttttttttttttttttttttttttttttttttttttnccncctnttaagtntttattananncccatngggtnccctntccccgntttgtatccctgccctgngggcctnanagccanatntcctganagcanagngaaaaggaanctcantntttntnaggcaggccancaggtcggaagggatnggggtttttgccactcctgccccattcattggcagcctgatcagccagngagtcctccgcnccatggccaaanaatctntggatnttntttttggcatcgttgatcacttttgcagcccaggcncccccaggtccccttttggcagnataatagttcccccgagcatggaantatttntctgacccnangtaaattgggcttnntttatgtcaanagagggctttcacatgtcccgagcccccatnaaaaaagcctcgccaagggnacnaaaanaancttttggntggtggcnccccggnaccnagggggcaaaaaacccgcccccgngaaaaancttnaatggggcntnatcttgggctngaaccttnaaccgnccggaccccctnnggttccancccccccttttttttttnggggttnggggggggggtttttttcntcaccggaaananaaagggntttatatanccccnncccttgggggggggggattaaaaaacnccccngggggggattnccaaaccccntttnancccangttnggnncccccccncccgggggacanggggttttnnaatanaac

SEQ ID NO: 12 - DNA, 905 bp, Homo sapiens
  misc_feature 3, 6, 7, 217, 230, 231, 232, 237, 238, 247, 249, 258,259, 260, 264, 265, 270, 272, 273, 276, 277, 278, 281, 290, 295,299, 300, 304, 305, 306, 311, 312, 314, 315, 318, 321, 322,324, 327, 328, 329, 330, 343, 345, 346, 351, 352, 356: n = A,T,C or G
  misc_feature 357, 364, 377, 380, 382, 385, 386, 390, 391, 394, 395,397, 402, 404, 406, 411, 418, 420, 423, 428, 430, 432, 446, 449,459, 463, 465, 468, 479, 497, 501, 505, 521, 531, 543, 552,553, 555, 570, 572, 573, 575, 593, 594, 601, 605, 609: n = A,T,C or G
  misc_feature 614, 620, 621, 627, 629, 641, 646, 674, 682, 693, 698,706, 726, 734, 739, 748, 750, 758, 769, 776, 777, 792, 793, 800,816, 821, 837, 838, 840, 841, 848, 856, 868, 878, 879, 884,891, 892, 897, 900, 902: n = A,T,C or G
ttnatnnaatcaagccgacaaccttgattggagacttgaccaaacctctggcgaagaagtccaaagctccaccgcggtggcggccgttacttacttagagctcgacgtcttacttacttagcggccgcccttttttttttttttttttttttttttttttttttttttttttttttttttttttttttttttttttttttttttttttttttttttncccccctttaaannnttaannaaaaccccntngggcccccnnncccnnttttntnnccnnnccnggggggccnaaaanccanntttnnngaaannannggnaanngnaannnntttttttttaagncnncccannaggnngaaaggnatgggggtttttnccncnccnncccnnttnntnggcancntnaaaanccagggngnccnccccncnanggcaaaaaaatttnggnattttttttngggnanggnggaaaaatttnggaccccaaagccccccnaggncccnttttggaaaaaaaaantttccccccnaaaaaggaaaantttttttcnnanaacccaaaaaaaaantnncntttttttaaaggggaaannaaagggntttncccnaaatnttcccnncccccnanaaaaaaaacccnctccnaaaaaaaaaaaaaaaaaaaaatttttgngtttgttngcccccccggncccangggggggnaaaaaacccccccccccggnaaaaaacnttaangggggccananttttggcntaaaaatccgnacccccnngggggcaaccccctnngtcccancccccctttttttttnggggnggggggggggttttcnncnncgaaaanggggggtnaacccccccccngggggggggnnaaaancccccgnnggaanccnanccc

SEQ ID NO: 13 - protein, 189 aa, Homo sapiens
MESAEEPLPARPALETEGLRFLHVTVGSLLASYGWYVLFSCILLYIVIQKLSVRLRVLRQRQLDQADAVLEPDAVVKRQEALAAARLRMQEDLNAQVEKHKEKLRQLEEEKRRQKIEMWDSMQEGRSYRRNPGRPQEEDGPGPSTSSSVTRKGKSDKKPLRGNGYNPLTGEGGGTCAWRPGRRGPSSGG

SEQ ID NO: 14 - protein, 37 aa, Homo sapiens
MESAEEPLPARPALETEGLRFLHVTVGSLLASYGWYV

SEQ ID NO: 15 - protein, 137 aa, Homo sapiens
VRLRVLRQRQLDQADAVLEPDAVVKRQEALAAARLRMQEDLNAQVEKHKEKLRQLEEEKRRQKIEMWDSMQEGRSYRRNPGRPQEEDGPGPSTSSSVTRKGKSDKKPLRGNGYNPLTGEGGGTCAWRPGRRGPSSGG

SEQ ID NO: 16 - protein, 73 aa, Homo sapiens
EMWDSMQEGRSYRRNPGRPQEEDGPGPSTSSSVTRKGKSDKKPLRGNGYNPLTGEGGGTCAWRPGRRGPSSGG

SEQ ID NO: 17 - DNA, 1152 bp, P. obesus
tcgttggtttcggcggccatggagagcgcagaggagcctctgcccgcgcggccggcgctggagaccgagggcctgaggttcctgcacgtcacagtgggctccctgctggccagctatggctggtacgtcctcttcagctgcatccttctctacattgtcatccagaagctctccgtccgattgagggttttgaggcagaggcagctggaccaggctgacgctgttctggaacctgatgctgttgttaagcgacaagaggctttagccgctgctcgtttgagaatgcaggaagatctaaatgcccaagttgaaaagcataaggaaaaactaagacagcttgaagaagaaaaaaggagacagaagattgaaatgtgggacagcatgcaagaaggcagaagttacagaagaaatccaggaaggcctcaggaagaagatggtcctggaccttctacttcatcatctgtcacccgcaaaggaaaatctgacaaaaagcctttgaggggaaatggttataaccctctgacgggtgaagggggtggaacctgcgcctggagacctggacgcaggggcccatcatctggtggatgaagctaagacccttgttagtgtcgctttgacattagcaaggtgaacccttaaccctcaactcagttgccttacgcacactttcacagtgactagccaaggagaggtggggcttatttccattcgtagctacctgtattctaagggctttggtcagtgtgagctatggacattgtcattaggtcatattctacttagacaacagtcattgatttcatggctacttgctagttgataggttaaaggcctctcgctgtttagcaaacttcataaaggaggcccagtgatgatcctttggggtagaagtccttgctgacaggatggtctctgtgacaggatgcgttcaatgatgtcttccttataaatggtgagcccaccagtgaggattactgatgtgcacagttgatggggtttgcttctgtatatttatttttatgtacagaaatttgcaaaaaaaaataaaaagtaacatttttagcatctttattaaactcaaggaaatttcgttgtgagcttgactttgtctatcagacattaaacagctttttatcattaaaaaaaaaaaaa

SEQ ID NO: 18 - protein, 189 aa, P. obesus; identical to SEQ ID NO: 13

SEQ ID NO: 19 - protein, 189 aa, P. obesus; identical to SEQ ID NO: 13

SEQ ID NO: 20 - protein, 16 aa, Homo sapiens
DRDSSRLAETVKSPGS

SEQ ID NO: 21 - protein, 36 aa, Mus musculus
RQSLSTTIVVFAARAAVVKEKKKGAKKSLKRRGSAS

SEQ ID NO: 22 - DNA, 23 bp, Artificial Sequence
  tanis gene forward primer
gatgcgttcaatgatgtcttcct

SEQ ID NO: 23 - DNA, 21 bp, Artificial Sequence
  tanis gene reverse primer
gaagcaaaccccatcaactgt

SEQ ID NO: 24 - DNA, 22 bp, Artificial Sequence
  beta-actin forward primer
gcaaagacctgtatgccaacac

SEQ ID NO: 25 - DNA, 23 bp, Artificial Sequence
  beta-actin reverse primer
gccagagcagtgatctctttctg

SEQ ID NO: 26 - DNA, 29 bp, Artificial Sequence
  fluorogenic probe sequence primer
cacatcagtaatcctcactggtgggctca

SEQ ID NO: 27 - DNA, 22 bp, Artificial Sequence
  tanis gene primer
tgctggcaccagacttgccctc

SEQ ID NO: 28 - DNA, 22 bp, Artificial Sequence
  PCR vector-specific forward primer
gaataagtgcgacatcatcatc

SEQ ID NO: 29 - DNA, 22 bp, Artificial Sequence
  PCR vector-specific reverse primer
gtaaatttctggcaaggtagac

SEQ ID NO: 30 - DNA, 15 bp, Artificial Sequence
  G-anchor primer
aagcttttttttttg

SEQ ID NO: 31 - DNA, 13 bp, Artificial Sequence
  arbitrary primer
aagcttctcaacg

(19) United States
(12) Patent Application Publication
(10) Pub. No.: US 2008/0039364 A1
(43) Pub. Date: Feb. 14, 2008

(54) Modulating serum amyloid a interaction with tanis and agents useful for same

(75) Inventors: Greg Collier, Ocean Grove (AU); Ken Walder, Ocean Grove (AU); Paul Zimmet, Toorak (AU); Lyndal Jane Bayles, Rippleside (AU); Yuan Gao, Highton (AU)

Correspondence Address: SEED INTELLECTUAL PROPERTY LAW GROUP PLLC, 701 FIFTH AVE, SUITE 5400, SEATTLE, WA, 98104, US

(73) Assignee: Autogen Research Pty Ltd, South Melbourne (AU)

(21) Appl. No.: 11/707,300

(22) Filed: Feb. 16, 2007

Related U.S. Application Data
(63) Continuation of application No. 10/481,652, filed on Jul. 8, 2004, now abandoned.

(30) Foreign Application Priority Data
Jun. 22, 2001 (AU) PR5898/01

(51) Int. Cl. A61K 48/00; G01N 33/53

(52) U.S. Cl. 514/2; 435/7.1

(57) ABSTRACT

The present invention relates generally to a method of modulating the functional activity of a serum amyloid A or derivative, homologue, analogue, equivalent or mimetic thereof and, more particularly, to a method of modulating the functional activity of a serum amyloid A by modulating intracellular levels of Tanis or derivative, homologue, analogue, chemical equivalent or mimetic thereof. The method of the present invention is particularly useful, inter alia, in the treatment and/or prophylaxis of conditions characterised by aberrant, unwanted or otherwise inappropriate serum amyloid A activity. The present invention is further directed to methods for identifying and/or designing agents capable of modulating Tanis mediated regulation of a serum amyloid A functional activity.

### CROSS-REFERENCE TO RELATED APPLICATIONS

This application is a continuation of U.S. patent application Ser. No. 10/481,652, filed Jul. 8, 2004, now pending, which application is a U.S. national stage application of PCT/AU2002/00815, international filing date of Jun. 21, 2002, which applications are incorporated herein by reference in their entireties.

### FIELD OF THE INVENTION

The present invention relates generally to a method of modulating the functional activity of a serum amyloid A or derivative, homologue, analogue, equivalent or mimetic thereof and, more particularly, to a method of modulating the functional activity of a serum amyloid A by modulating intracellular levels of Tanis or derivative, homologue, analogue, chemical equivalent or mimetic thereof. The method of the present invention is particularly useful, inter alia, in the treatment and/or prophylaxis of conditions characterized by aberrant, unwanted or otherwise inappropriate serum amyloid A activity. The present invention is further directed to methods for identifying and/or designing agents capable of modulating Tanis mediated regulation of a serum amyloid A functional activity.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

The serum amyloid A (herein referred to as “SAA”) proteins are a family of acute phase proteins which are upregulated in response to inflammation. SAA is the collective name given to this family which are also polymorphic and are encoded by multiple genes (Bausserman et al., 1980; Kluve-Beckerman et al., 1986; Kluve-Beckerman et al., 1991).

Extensive analyses have revealed the SAA superfamily to be a cluster of closely linked genes localized to human chromosome 11p15 (Seller et al., 1994). Watson et al (1994) further demonstrated that all of the functional genes of the SAA superfamily (ie. SAA1, SAA2 and SAA4) map within the region SAAs are small apolipoproteins that associate rapidly during the acute phase response with the third fraction of high-density lipoprotein (HDL3), on which they become the predominant apolipoprotein. SAA enhances the binding of HDL3 to macrophages during inflammation, concomitant with a decrease in the binding capacity of HDL3 to hepatocytes (reviewed in Jensen and Whitehead 1998). These changes suggest that SAA may remodel HDL3 and act as a signal to redirect it from hepatocytes to macrophages, which can then engulf cholesterol and lipid debris at sites of necrosis. In this way, excess cholesterol can be redistributed for use in tissue repair or excreted.

Increased acute phase response proteins, including SAA, are detected in type 2 diabetics. Increased SAA in type 2 diabetes may act to redirect HDL cholesterol from the liver to the macrophage for tissue repair. This increased catabolism is thought to be a possible reason for the low HDL concentrations observed in diabetic patients, and the uptake by macrophages in the atherosclerotic plaque could be part of the reason for an increased risk of arterial disease in type 2 diabetics.

SAA levels can increase by as much as 1000-fold in response to injury, infection or inflammation and secondary, or reactive amyloidosis is one consequence of a variety of chronic and recurrent inflammatory diseases. Secondary amyloid deposits are comprised mainly of amyloid A, thought to be derived by proteolysis from the precursor SAA. Upon cleavage from the parent product, amyloid A can aggregate into insoluble antiparallel beta-pleated sheet fibrils which cause the systemic complications known as amyloidosis (Falk et al., 1997). By definition, amyloid fibrils stain positive with Congo Red and exhibit green bi refringence when viewed with polarised light (Behold, 1922).

Serum amyloid A proteins are well conserved throughout evolution and have been implicated in a range of other disease states including arthritis, multiple sclerosis, scleroderma, trauma, ankylosing spondylitis, colitis, acute pancreatitis, transplant rejection, infection and heart disease.

Accordingly, elucidation of the mechanisms of action of the serum amyloid A proteins is necessary for the development of therapeutic and/or prophylactic strategies directed to treating conditions which are characterised by aberrant or otherwise unwanted serum amyloid functional activities.

In work leading up to the present invention, the inventors have determined that the Tanis protein interacts with serum amyloid A proteins. The expression of Tanis had previously been thought to be essentially regulated by fasting and feeding thereby providing a mechanism for regulating body weight and energy metabolism. Without limiting the present invention in any way, Tanis is thought to exist as a membrane bound protein and to function as a receptor. Identification of the interaction between Tanis and serum amyloid A has significantly broadened the current understanding in relation to the functional role of Tanis and has now facilitated the development of methodology directed to modulating serum amyloid A mediated functional activity. Further, there is facilitated the design of therapeutic and/or prophylactic regimes for treating conditions characterised by aberrant, unwanted or otherwise inappropriate serum amyloid A functional activity.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide and amino acid sequence information prepared using the program PatentIn Version 3.0, presented herein after the bibliography. Each nucleotide or amino acid sequence is identified in the sequence listing by the numeric indicator <210> followed by the sequence identifier (e.g. <210> 1, <210> 2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (eg. <400> 1, <400> 2, etc).

One aspect of the present invention provides a method of modulating the functional activity of an apolipoprotein or derivative, homologue, analogue, chemical equivalent or mimetic thereof in a subject, said method comprising administering to said subject an effective amount of an agent for a time and under conditions sufficient to modulate the interaction of Tanis or derivative, homologue, analogue, chemical equivalent or mimetic thereof with said apolipoprotein.

In another aspect, there is provided a method of modulating the functional activity of a SAA or derivative, homologue, analogue, chemical equivalent or mimetic thereof in a subject, said method comprising administering to said subject an effective amount of an agent for a time and under conditions sufficient to modulate the interaction of Tanis or derivative, homologue, analogue or chemical equivalent thereof with said SAA.

In yet another aspect there is provided a method of modulating the functional activity of Tanis or derivative, homologue, analogue, chemical equivalent or mimetic thereof in a subject, said method comprising administering to said subject an effective amount of an agent for a time and under conditions sufficient to modulate the interaction of an apolipoprotein or derivative, homologue, analogue, chemical equivalent or mimetic thereof with said Tanis.

In still another aspect, there is provided a method of modulating the functional activity of Tanis or derivative, homologue, analogue, chemical equivalent or mimetic thereof in a subject, said method comprising administering to said subject an effective amount of an agent for a time and under conditions sufficient to modulate the interaction of a SAA or derivative, homologue, analogue, chemical equivalent or mimetic thereof with said Tanis. Another aspect of the present invention is directed to a method for the treatment and/or prophylaxis of a condition characterised by aberrant, unwanted or otherwise inappropriate apolipoprotein mediated functional activity in a mammal, said method comprising administering to said mammal an effective amount of an agent for a time and under conditions sufficient to modulate the interaction of Tanis with said apolipoprotein.

Still another aspect of the present invention contemplates the use of an agent, as hereinbefore defined, in the manufacture of a medicament for the treatment of a condition in a mammal, which condition is characterised by aberrant, unwanted or otherwise inappropriate SAA mediated cellular activity, wherein said agent modulates the interaction of Tanis with an SAA.

In another aspect, the present invention contemplates the use of an agent, as hereinbefore defined, in the manufacture of a medicament for the treatment of a condition in a mammal, which condition is characterised by aberrant, unwanted or otherwise inappropriate Tanis mediated functional activity, wherein said agent modulates the interaction of Tanis with an SAA.

In yet another further aspect, the present invention contemplates a pharmaceutical composition comprising the modulatory agent as hereinbefore defined together with one or more pharmaceutically acceptable carriers and/or diluents.

Yet another aspect of the present invention relates to the agent as hereinbefore defined, when used in the method of the present invention.

Another aspect of the present invention provides a method for detecting an agent capable of modulating the interaction of Tanis with SAA or its derivative, homologue, analogue, chemical equivalent or mimetic thereof said method comprising contacting an in vitro system containing said Tanis and SAA with a putative agent and detecting an altered expression phenotype associated with said interaction.

Single and three letter abbreviations used throughout the specification are defined in Table 1.

TABLE 1
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | The | T |
| Tryptophan | Trp | W |
| Tyrosine | Tyr | Y |
| Valine | Val | V |
| As defined | Xaa | X |

### BRIEF DESCRIPTION OF THE DRAWINGS

FIG. 1 is a graphical representation of a typical sensorgram showing negative and positive binding results.

FIG. 2 is a graphical representation of a sensorgram showing full length GST-Tanis interacting with serum amyloid A bound to the CN5 chip.

Reference Points

1 baseline, inject 5 μl GST-FLtanis over 6 min (baseline and injection point)

2, 4, 6 maximum change in resonance during injection (max RU)

3, 5, 7 injections of 5 μl GST FLtanis over 6 min (injection points)

FIG. 3 is a graphical representation of a sensorgram showing GST-Tanis C-terminal only interacting with the SAA bound to the CN5 chip.

Reference Points

1, 8 regeneration of the chip, stripping of any proteins interacting with SAA

2, 4, 6 injection of Tanis Cplus protein

3, 5, 7 maximum response (interaction) during injection

FIG. 4 is an image of: A: Autoradiograph of ddPCR gel showing upregulated Tanis gene expression in the liver of P. obesus in the fasted state (arrow shows location of band corresponding to the Tanis gene). B: Nucleotide (<400>17) and amino acid (<400>18) sequences of the P. obesus Tanis gene. Putative transmembrane sequence is underlined. C: Amino acid sequence (<400>19) of the P. obesus Tanis gene aligned with corresponding genes from human (AD-015) (<400>20) and mouse (H47) (<400>21). D: Genomic structure of the P. obesus Tanis gene.

FIG. 5 is a graphical representation of hepatic Tanis gene expression in P. obesus. A: Reduced Tanis gene expression in the liver of IGT and type 2 diabetic P. obesus in the fed state. B: Fold increase in Tanis gene expression in the liver of P. obesus after 24-h fast compared with the fed state. *Significantly different from the nGT group (P<0.05); **significant increase compared with the fed diabetic group (P=0.010).

FIG. 6 is a graphical representation of the linear correlation between hepatic Tanis gene expression and circulating triglyceride concentrations in P. obesus (r=0.593, P=0.007).

FIG. 7 is a graphical representation of the effects of increasing glucose concentration on Tanis gene expression in HepG2 hepatocytes. *Significantly different from glucose concentration of 0 mmol/l (P<0.001).

FIG. 8 is an image of Northern blot for Tanis in P. obesus. Lane 1, size marker; lane 2, adipose tissue; lane 3, hypothalamus; lane 4, liver; lane 5, skeletal muscle. Arrow indicates position of the Tanis gene.

FIG. 9 is a graphical representation of the effects of glucose (upper panel) and insulin (lower panel) concentrations on the expression of the Tanis gene in 3T3-L1 adipocytes. *Significantly different from glucose concentration of 0 mmol/l (P<0.001); **significantly different from insulin concentration of 0 nmol/l. (P=0.020).

FIG. 10 is a graphical representation of the real-time interaction between Tanis and SAA by SPR analysis. The ligand (A and B, human plasma SAA; C, GST-Tanis-C; D, GST-SAA) was immobilized onto the CM5 sensor chip and the analyte (A, B, and D, GST-Tanis-C, 5 μg; C, human plasma SAA 5 μg) diluted in binding buffer was passed over the chip. The change in SPR was indicated in Ru. The samples were injected over 4 (A, C, and D) or 6 (B) min. The injection points are indicated by arrows. Injection of GST control protein alone (A, B, and D) did not produce a binding phenomenon.

FIG. 11 is an image of the fractionation and western blot of sand rat liver (left) and fat tissues (right). These tissues were fractionated into mitochondria/nuclei (M/N), plasma membrane (PM), high-density microsomes (HDM), low-density microsomes (LDM) and soluble (Sol) proteins and probed with anti-Tanis-C antibody. The fractionation procedure involved homogenzing the tissues in a glass douncer, a low speed spin (2000 g×15 min) to pellet the M/N (P1) from the supernatant (S1). The S1 was spun at 18,000 g×15 min resulting in a pellet (P2) and supernatant S2. The pellet P2 was further purified over a sucrose gradient cushion to obtain PM. The S2 fraction was spun sequentially at 100,000 g×70 min to yield HDM and 200,000 g for LDM fractions. The supernatant after the last spin was designated as soluble proteins.

FIG. 12 is an image of Tanis gene expression (A) and protein levels (B) during feeding and fasting (24 h) in the sand rats. The gene expression data in panel A have been presented in previous “Quarterly Report”, and are included here for the sole purpose for comparison with the protein levels. In panel B, plasma membrane and microsomes (containing both high- and low-density microsomes) were isolated from the liver and fat of three fed or fasted diabetic/obese sand rats. Tanis protein in each animal was visualized in western blots with the anti-Tanis-C antibody.

FIG. 13 is a representation of Tanis gene expression and protein levels being enhanced by low glucose in HepG2 cells. Cells were grown in DMEM (25 mM glucose) and 10% FBS. The cells were then treated with varying concentrations of glucose (0.5-25 mM) in DMEM for 24 h. Total RNA was extracted from the cells and Tanis transcript was quantified by reverse transcription and real time PCR. Tanis protein was detected in western blot using the anti-Tanis-C antibody.

FIG. 14 is a graphical representation of glycogen content in Tanis-expressing H4IIE cells. H4IIE cells were infected with adenovirus expressing Tanis or GFP or without virus. Forty hours after infection, cells were treated with insulin for 6 h and harvested. Glycogen was determined by first digesting with amyloglucosidase, and the released glucose was assayed enzymatically by hexokinase and glucose-6-phosphate dehydrogenase coupled with the reduction of NADP. The values presented are absorbance at 340 nm per well of cells, which had reached confluency at the time of harvest in all treatments.

FIG. 15 is a graphical representation depicting glycogen synthesis in H4IIE cells. H4IIE cells were grown in 6-well plates and infected with or without adenovirus expressing Tanis or GFP. 30 h post infection, cells were serum-starved in DMEM (5.5 mM glucose) overnight. Cells were then incubated in DMEM (5.5 mM glucose) containing ¹⁴C-glucose (1 μCi/mL) for three hours, lysed in 30% KOH. Glycogen was precipitated by acetone and counted for ¹⁴C by liquid scintillation. The values presented are DPM per well of cells, which had reached confluency at the time of harvest in all treatments.

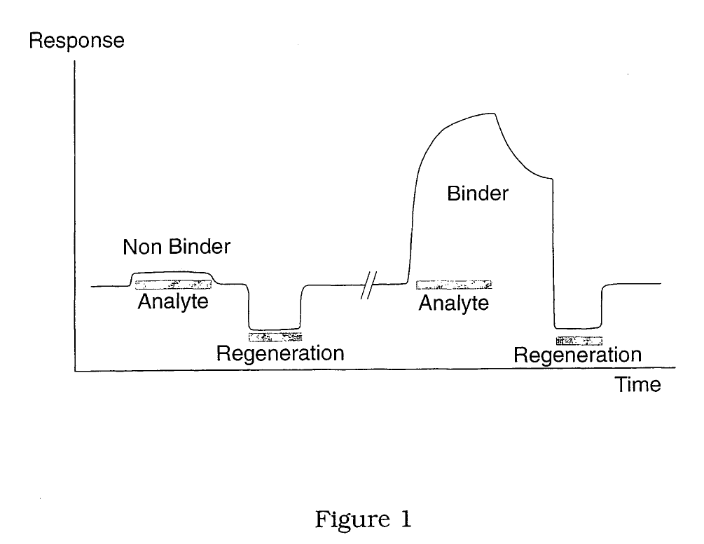

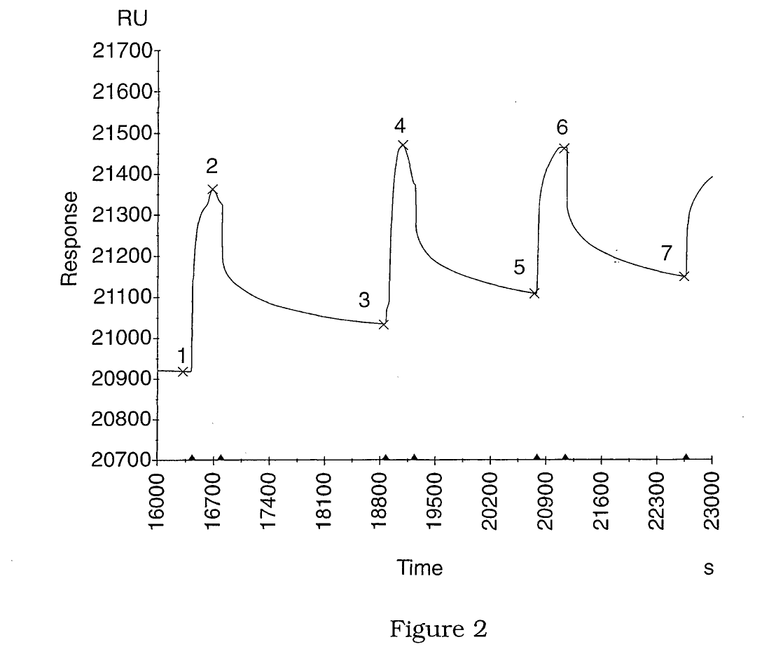

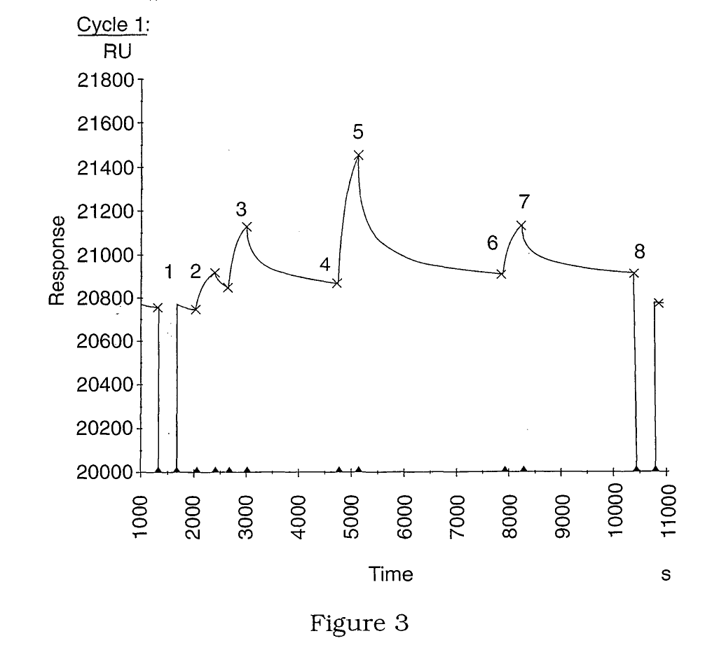

### DETAILED DESCRIPTION OF THE INVENTION

The present invention is predicated, in part, on the elucidation of an interactive relationship between Tanis and the serum amyloid A proteins. This determination now permits the rational design of therapeutic and/or prophylactic methods for treating conditions characterised by unwanted serum amyloid A activity. Further, there is facilitated the identification and/or design of agents which modulate Tanis mediated regulation of serum amyloid A functional activity.

Accordingly, one aspect of the present invention provides a method of modulating the functional activity of an apolipoprotein or derivative, homologue, analogue, chemical equivalent or mimetic thereof in a subject, said method comprising administering to said subject an effective amount of an agent for a time and under conditions sufficient to modulate the interaction of Tanis or derivative, homologue, analogue, chemical equivalent or mimetic thereof with said apolipoprotein.

More particularly, there is provided a method of modulating the functional activity of a SAA or derivative, homologue, analogue, chemical equivalent or mimetic thereof in a subject, said method comprising administering to said subject an effective amount of an agent for a time and under conditions sufficient to modulate the interaction of Tanis or derivative, homologue, analogue or chemical equivalent thereof with said SAA.

Reference to “SAA” should be understood as a reference to any member of the SAA superfamily of acute phase proteins. Without limiting the present invention to any one theory or mode of action, SAAs are small apolipoproteins. Their levels are increased markedly during infection, inflammation or after injury and they are known to associate with the third fraction of high density lipoproteins thereby remodelling HDL3 and enhancing binding of HDL3 to macrophages during infection. With respect to each member of the SAA family, the term “SAA” should also be understood to encompass all forms of that member or derivative, homologue, analogue, chemical equivalent or mimetic thereof. It should also be understood to include reference to any isoforms which arise from alternative splicing of SAA mRNA or mutants or polymorphic variants of SAA. In this regard, the SAA family of proteins are known to be polymorphic and encoded by multiple genes. “SAA” should further be understood to include reference to any other molecules which exhibit at least one SAA functional activity.

Reference to “SAA mediated functional activity” should be understood as a reference to any one or more of the functional activities, such as physiological processes or cellular activities, which are directly or indirectly induced by the actions of SAA. A “directly” induced functional activity should be understood as reference to an activity which is initially induced as a result of a SAA signal (for example, the re-direction of HDL3 from hepatocytes to macrophages) or interaction with SAA (for example, re-modelling of HDL3 following its binding to SAA). “Indirectly induced activities” should be understood as those activities which are a downstream consequence of a direct action as hereinbefore defined. For example, induction of SAA is known to be associated with increased DNA binding activities of at least 3 transcription factors, Nuclear Factor-kappa B, CCAAT enhancer binding protein and SAA-Activating Factor (Ray and Ray, 1999). These transcription factors are not specific for SAA and they are known to alter the transcription of a number of other genes. Activation of these factors may therefore indirectly alter the expression of these genes by altering the binding activity of the above identified transcription factors.

Reference to “Tanis” or “nucleotide sequence encoding Tanis” should be understood as a reference to all forms of Tanis or derivative, homologue, analogue, chemical equivalent or mimetic thereof or other molecules having the function of Tanis. This includes, for example, all protein or nucleic acid forms of Tanis or its functional equivalent or derivative, including, for example, any isoforms which arise from alternative splicing of Tanis mRNA or mutants or polymorphic variants of Tanis. It should be understood that reference to a “nucleotide sequence encoding Tanis” includes reference to any Tanis regulatory element (such as promoters or enhancers) which regulates the expression of Tanis and include the location at a position other than between the Tanis genomic DNA transcription initiation and termination sites. “Tanis” should also be understood to include reference to any other molecules which exhibit the functional activity of Tanis. Such molecules include, for example, endogenously expressed molecules which exhibit Tanis functional activity or molecules which have been introduced into the body and which mimic at least one of the Tanis functions. These molecules may be recombinant, synthetic or naturally occurring. To the extent that it is not specified, any reference to modulating the expression of a nucleic acid molecule encoding Tanis or the functional activity of the Tanis expression product should be understood to include reference to modulating the expression or functional activity of Tanis functional equivalents or derivatives. Without limiting the present invention in any way, Tanis is also known as “band 55” or “B55”, the nucleic acid cDNA and genomic sequences of which are provided in International Patent Publication No. WO01/02560, which is incorporated herein by reference. Psammomys obesus Tanis comprises the sequence set forth in <400>13. It should be understood that a genomic sequence may also comprise exons and introns. A genomic sequence may also include a promoter region or other regulatory region. It should be understood that the genomic sequence disclosed in International Patent Publication No. WO01/02560 corresponds only to that part of the sequence running from the transcription initiation site to the transcription termination site. Accordingly, this sequence and other genomic sequences encompassed by the present invention may comprise either more or less sequence than encompassed from the transcription initiation site to the transcription termination site. For example, it may comprise additional non-translated sequences such as regulatory sequences located up or downstream of the transcription site/sites. Reference to nucleic acid molecules encoding Tanis are herein indicated in italicised text as Tanis.

Without limiting the present invention to any one theory or mode of action, it has been determined that Tanis interacts with SAAs. Tanis is thought to exist as a membrane bound molecule which functions as a receptor for SAAs. Accordingly, it is thought that SAA signals a cell via its interaction with Tanis. Elucidation of the existence of a Tanis-SAA interactive relationship now provides a mechanism for modulating SAA mediated cellular activities and/or physiological processes. By “modulation” is meant up or down regulation. It should be understood that modulation of the interaction between Tanis and a SAA (either in the sense of up regulation or down regulation) may be partial or complete. Partial modulation occurs where only some of the SAA-Tanis interactions which would normally occur in a given subject are affected by the method of the present invention (for example, the method of the present invention is applied to a subject for only part of the time that the cell is undergoing SAA mediated functional activity or the agent which modulates the interaction of Tanis with SAA is provided in a concentration insufficient to saturate all Tanis-SAA interactions) while complete modulation occurs where all Tanis-SAA interactions are modulated.

Although the preferred method is to modulate SAA mediated functional activity via modulation of the SAA-Tanis interaction, it is also feasible to modulate Tanis-mediated functional activities, particularly to the extent that Tanis may also function in a non-membrane bound form, via modulation of its interaction with a SAA.

Accordingly, in another aspect there is provided a method of modulating the functional activity of Tanis or derivative, homologue, analogue, chemical equivalent or mimetic thereof in a subject, said method comprising administering to said subject an effective amount of an agent for a time and under conditions sufficient to modulate the interaction of an apolipoprotein or derivative, homologue, analogue, chemical equivalent or mimetic thereof with said Tanis.

More particularly, there is provided a method of modulating the functional activity of Tanis or derivative, homologue, analogue, chemical equivalent or mimetic thereof in a subject, said method comprising administering to said subject an effective amount of an agent for a time and under conditions sufficient to modulate the interaction of a SAA or derivative, homologue, analogue, chemical equivalent or mimetic thereof with said Tanis.

Reference to “Tanis mediated functional activity” should be understood as a reference to any one or more functional activities, such as cellular activities or cellular signalling mechanisms, which are directly or indirectly induced via the actions of Tanis. Reference to “direct” and “indirect” actions should have the same general meaning as hereinbefore defined in relation to SAA mediated functional activity.

Modulation of the interaction between Tanis and SAA may be achieved by any one of a number of techniques including, but not limited to:
(i) introducing into a cell a nucleic acid molecule encoding Tanis or SAA or derivative, homologue or analogue thereof or introducing into a subject the proteinaceous form of Tanis or SAA or derivative, homologue, analogue, chemical equivalent or mimetic thereof in order to modulate the intracellular concentrations of Tanis or SAA which are available for interactive purposes.
(ii) introducing into a cell a proteinaceous or non-proteinaceous molecule which modulates the transcriptional and/or translational regulation of a gene, wherein said gene may be a Tanis gene or an SAA gene.
(iii) introducing into a cell a proteinaceous or non-proteinaceous molecule which antagonises the interaction between Tanis and a SAA.
(iv) introducing into a cell a proteinaceous or non-proteinaceous molecule which agonises the interaction between Tanis and a SAA.

Reference to “agent” should be understood as a reference to any proteinaceous or non-proteinaceous molecule which modulates the interaction of Tanis with a SAA and includes, for example, the molecules detailed in points (i)-(iv), above. The subject agent may be linked, bound or otherwise associated with any proteinaceous or non-proteinaceous molecule. For example, it may be associated with a molecule which permits its targeting to a localised region.

Said proteinaceous molecule may be derived from natural, recombinant or synthetic sources including fusion proteins or following, for example, natural product screening. Said non-proteinaceous molecule may be derived from natural sources, such as for example natural product screening or may be chemically synthesised. The present invention contemplates chemical analogues of said Tanis or SAA capable of acting as agonists or antagonists of the Tanis-SAA interaction. Chemical agonists may not necessarily be derived from said Tanis or SAA but may share certain conformational similarities. Alternatively, chemical agonists may be specifically designed to mimic certain physiochemical properties of said Tanis or SAA. Antagonists may be any compound capable of blocking, inhibiting or otherwise preventing said Tanis and SAA from interacting. Antagonists include monoclonal antibodies specific for said Tanis or SAA, or parts of said Tanis, and antisense nucleic acids which prevent transcription or translation of genes or mRNA in the subject cells. Modulation of expression may also be achieved utilising antigens, RNA, ribosomes, DNAzymes, RNA aptamers, antibodies or molecules suitable for use in co-suppression. Screening methods suitable for use in identifying such molecules are described in more detail hereinafter.

Said proteinaceous or non-proteinaceous molecule may act either directly or indirectly to modulate the interaction of Tanis with SAA. Said molecule acts directly if it associates with the Tanis or SAA molecules. Said molecule acts indirectly if it associates with a molecule other than Tanis or SAA, which other molecule either directly or indirectly modulates the interaction of Tanis with SAA. Accordingly, the method of the present invention encompasses regulation of the Tanis-SAA interaction via the induction of a cascade of regulatory steps.

“Derivatives” include fragments, parts, portions, mutants, variants and mimetics from natural, synthetic or recombinant sources including fusion proteins. Parts or fragments include, for example, active regions of Tanis or SAA. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Reference to “homologues” should be understood as a reference to nucleic acid molecules or proteins derived from species other than the species being treated.

Chemical and functional equivalents of nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

It should be understood that although the preferred method of the present invention is to treat a subject, the method of the present invention may also be adapted for application in vitro. Such adaptation could be routinely performed by the person of skill in the art. For example, to the extent that it is sought to modulate cellular activity which is mediated via the Tanis membrane bound molecule, it is feasible that interaction with SAA or a modulatory agent as hereinbefore defined could be performed in vitro. This may be desirable, for example, where it is sought to establish an in vitro system for screening for molecules which modulate the SAA-Tanis interaction. Alternatively, it may be desirable to treat an in vitro population of cells according to the methods defined herein prior to their introduction to a subject. These cells may have been initially isolated from the subject, for example, and then returned following appropriate treatment.

A further aspect of the present invention relates to the use of the invention in relation to the treatment and/or prophylaxis of disease conditions. Without limiting the present invention to any one theory or mode of action, the pleiotropic activities of SAA render these molecules an integral functional component of many aspects of both healthy and disease state physiological processes. Accordingly, the method of the present invention provides a valuable tool for modulating aberrant or otherwise unwanted SAA functional activity. In a related aspect, the present invention also provides a tool for modulating aberrant or otherwise unwanted Tanis functional activity, to the extent that this functional activity is mediated by SAA.

Accordingly, another aspect of the present invention is directed to a method for the treatment and/or prophylaxis of a condition characterised by aberrant, unwanted or otherwise inappropriate apolipoprotein mediated functional activity in a mammal, said method comprising administering to said mammal an effective amount of an agent for a time and under conditions sufficient to modulate the interaction of Tanis with said apolipoprotein.

More particularly, there is provided a method for the treatment and/or prophylaxis of a condition characterised by aberrant, unwanted or otherwise inappropriate SAA mediated functional activity in a mammal, said method comprising administering to said mammal an effective amount of an agent for a time and under conditions sufficient to modulate the interaction of Tanis with said SAA.

Yet another aspect of the present invention is directed to a method for the treatment and/or prophylaxis of a condition characterised by aberrant, unwanted or otherwise inappropriate Tanis mediated functional activity in a mammal, said method comprising administering to said mammal an effective amount of an agent for a time and under conditions sufficient to modulate the interaction of apolipoprotein with said Tanis.

More particularly, there is provided a method for the treatment and/or prophylaxis of a condition characterised by aberrant, unwanted or otherwise inappropriate Tanis mediated functional activity in a mammal, said method comprising administering to said mammal an effective amount of an agent for a time and under conditions sufficient to modulate the interaction of SAA with said Tanis.

Reference to “aberrant, unwanted or otherwise inappropriate” functional activity should be understood as a reference to overactive functional activity, to physiologically normal functional activity which is inappropriate in that it is unwanted or to insufficient functional activity. For example, increased acute phase response proteins, including SAA, are detected in Type II diabetes. Increased SAA in Type II diabetes is thought to act to redirect HDL cholesterol from the liver to the macrophage for tissue repair. This increased catabolism is thought to be a possible reason for the low HDL concentrations observed in diabetic patients. Although a normal physiological process, the uptake by macrophages in the atherosclerotic plaque is thought to be part of the reason for an increased risk of arterial disease in Type II diabetics. Accordingly, in such a situation, it may be desirable to at least partially down-regulate the functional activity of SAA in order to alleviate some of the risk associated with development of arterial disease. In another example, it is known that SAA stimulation can lead to induction of collagenase, an enzyme known to be involved in tissue destruction often seen in inflammatory and proliferative rheumatoid arthritis and osteoarthritis (Mitchell et al., 1991; Brinckerhoff et al., 1989). Accordingly, in a situation such as this, it may be desirable to at least partially down-regulate the functional activity of SAA in order to alleviate some of the risk associated with the development of these diseases.

Preferably, said condition includes, but is not limited to:
(i) Arthritis—SAA is elevated in rheumatoid arthritis and SAA concentrations are thought to reflect disease severity (Grindulus et al., 1995; Cunnane and Whitehead, 1999; Cunnane et al., 2000).
(ii) Inflammatory conditions—SAA is elevated in a number of inflammatory conditions and circulating concentrations may represent a possible means of assessing the degree of inflammation. These conditions include multiple sclerosis (Ristori et al., 1998), scleroderma (Brandwein et al., 1984), trauma (Mozes et al., 1989), ankylosing spondylitis (Lange et al., 2000), colitis (Yang et al., 1999; de Villiers et al., 2000), acute pancreatitis (Pezzilli et al., 2000).
(iii) Transplantation—Pancreatic and renal transplantation rejection episodes have been related to increases in SAA (Casl et al., 1995; Hartmann et al., 1997; Muller et al., 1997; Kaysen et al., 1999). SAA appears to increase early in the rejection process and is thought to be a useful marker of the patient's inflammatory condition.
(iv) Infection—SAA has been shown to be a sensitive indicator of viral infections and acute diarrhoea (Whicher et al., 1985; Darling et al., 1999).
(v) Heart Disease—Increased concentrations of SAA are associated with increased risk of myocardial infarction (Liuzzi et al., 1994; Casl et al., 1995; Danesh et al., 1999), coronary heart disease (Stefanadis et al., 2000), coronary artery disease (Erren et al., 1999), cardiovascular disease (Ridket et al., 2000) and atherosclerosis (Meek et al., 1994; Erren et al., 1999).
(vi) Sarcoidosis (Salazar et al., 2000)
(vii) Alzheimer's disease (Chung et al., 2000)
(viii) Nephropathy (renal amyloidosis) (Kaneko et al., 2000) and end-stage renal disease (Kaysen et al., 1999)
(ix) Abdominal aortic aneuryism (Rhode et al., 1999)
(x) Obesity (Danesh et al., 1999)
(xi) Type 2 diabetes (Pickup et al., 1997; Pickup and Crook, 1998)
(xii) Any condition which involves aberrant immune responses related to Tanis or SAA or their interaction.

The term “subject” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the agent together with subjection of the mammal to insulin administration for the treatment of diabetes.

Administration of the modulatory agent, in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention contemplates the use of an agent, as hereinbefore defined, in the manufacture of a medicament for the treatment of a condition in a mammal, which condition is characterised by aberrant, unwanted or otherwise inappropriate apolipoprotein mediated functional activity, wherein said agent modulates the interaction of Tanis with an apolipoprotein.

Preferably, said apolipoprotein is SAA.

In another aspect, the present invention contemplates the use of agent, as hereinbefore defined, in the manufacture of a medicament for the treatment of a condition in a mammal, which condition is characterised by aberrant, unwanted or otherwise inappropriate Tanis mediated functional activity, wherein said agent modulates the interaction of Tanis with an apolopoprotein.

Preferably, said apolipoprotein is SAA.

In yet another further aspect, the present invention contemplates a pharmaceutical composition comprising the modulatory agent as hereinbefore defined together with one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to the agent as hereinbefore defined, when used in the method. of the present invention.

Screening for the modulatory agents hereinbefore defined can be achieved by any one of several suitable methods including, but in no way limited to, contacting a cell culture comprising Tanis and apolipoprotein, such as SAA with an agent and screening for the modulation of Tanis-SAA functional activity or modulation of the activity or expression of a downstream cellular target. Detecting such modulation can be achieved utilising techniques such as Western blotting, electrophoretic mobility shift assays and/or the readout of reporters of Tanis or SAA activity such as luciferases, CAT and the like.

It should be understood that the Tanis protein may be naturally occurring in the cell which is the subject of testing or the genes encoding them may have been transfected into a host cell for the purpose of testing. Further, the naturally occurring or transfected gene may be constitutively expressed—thereby providing a model useful for, inter alia, screening for agents which down-regulate Tanis-SAA interactivity or the gene may require activation—thereby providing a model useful for, inter alia, screening for agents which modulate Tanis-SAA interactivity under certain stimulatory conditions. Further, to the extent that a Tanis nucleic acid molecule is transfected into a cell, that molecule may comprise the entire Tanis gene or it may merely comprise a portion of the gene such as the SAA binding portion.

In another example, the subject of detection could be a downstream Tanis regulatory target, rather than Tanis itself. Yet another example includes Tanis binding sites ligated to a minimal reporter. For example, modulation of Tanis-SAA interactivity can be detected by screening for the modulation of the downstream signalling components of a SAA or Tanis stimulated cell. This is an example of a system where modulation of the molecules which Tanis and SAA regulate the activity of, are monitored.

Accordingly, another aspect of the present invention provides a method for detecting an agent capable of modulating the interaction of Tanis with apolipoprotein or its derivative, homologue, analogue, chemical equivalent or mimetic thereof said method comprising contacting an in vitro system containing said Tanis and apolipoprotein with a putative agent and detecting an altered expression phenotype associated with said interaction.

Preferably, said apolipoprotein is SAA.

Reference to “Tanis” and “SAA” should be understood as a reference to either the Tanis or SAA expression product or to a portion or fragment of the Tanis or SAA molecule, such as the SAA binding region of the Tanis protein. In this regard, to the extent that the Tanis or SAA expression product is expressed in a cell, the cell may be a host cell which has been transfected with the Tanis or SAA nucleic acid molecule or it may be a cell which naturally contains the Tanis gene.

Reference to detecting an “altered expression phenotype associated with said interaction” should be understood as the detection of cellular changes associated with modulation of the interaction of Tanis with SAA. These may be detectable, for example, as intracellular changes or changes observable extracellularly. For example, this includes, but is not limited to, detecting changes in downstream product levels or activities.

The present invention is further defined by the following non-limiting examples:

### EXAMPLE 1

### Yeast Two Hybrid Screening for Proteins Interacting with Tanis

(i) Materials and Methods

Plasmid Construction

The Tanis gene was amplified by PCR, from a housekeeping vector using gene specific primers that incorporated Sal I and Nco I restriction sites (Table 3). PCR products were gel purified using the QIAquick™ gel extraction kit (QIAGEN Pty. Ltd., Australia) before being subjected to restriction enzyme digestion with Sal I and Nco I (New England Biolabs Inc., Beverly, USA). Following digestion, samples were extracted once with phenol:chloroform:isoamyl alcohol (25:24:1), and DNA precipitated with 2 volumes of absolute ethanol and 0.1 volume of 3M sodium acetate pH 5.2. Digested PCR products were resuspended in 20 μl of nuclease free water and the relative concentration determined by agarose gel electrophoresis.

The yeast plasmid vector pDBLeu (Life Technologies Inc., USA) was similarly digested with Sal I and Nco I and the linearised products separated on a 1.0% agarose gel. Digested fragments were gel purified as described, and the concentration of purified vector DNA determined by agarose gel electrophoresis.

Following digestion, Tanis was ligated to the prepared pDBLeu vector DNA and the products transformed into DH5α (Life Technologies Inc., USA) by electroporation. Transformants were selected by growth on LB agar plates containing kanamycin at 25 μg/ml.

Recombinant clones were identified by means of colony PCR using vector specific primers (Table 1). Plasmid DNA from selected clones was then prepared and used as template for DNA sequencing. A positive clone, pDBB559, was selected for use in two-hybrid screening as sequencing revealed a 100% homologous Tanis gene sequence cloned in frame with the GAL4 DNA binding domain of pDBLeu.

TABLE 3
PCR cloning and sequencing primers

| | |
|---|---|
| Primer Name | Sequence |

| | | |
|---|---|---|
| ProB55F | 5′ ATCGAGTCGACCATGGAGA | <400>1 |
| Gene specific | GCGCAGAGGAGCCT 3′ | |
| forward | | |
| cloning primer | | |
| ProB55R | 5′ CAAGCCATGGCGCTTCATC | <400>2 |
| Gene specific | CACCAGATGATGG 3′ | |
| reverse | | |
| cloning primer | | |
| ProSeqF | 5′ GAATAAGTGCGACATCATC | <400>3 |
| pDBLeu forward | ATC 3′ | |
| vector specific | | |
| primer | | |
| ProSeqR | 5′ GTAAATTTCTGGCAAGGTA | <400>4 |
| pDBLeu reverse | GAC 3′ | |
| vector specific | | |
| primer | | |

Strain Construction

100 ng of pDBB559 was transformed into the yeast strain MaV203 (Life Technologies Inc., USA) using a standard, lithium acetate/polyethylene glycol procedure. Transformants containing the pDBB559 plasmid were selectively isolated by growth on plates lacking leucine.

3AT Titration

To determine basal levels of HIS3 expression, induced by GAL4 DB-Tanis, the activation domain vector pPC86 was introduced into MaV203 cells containing the pDBB559 plasmid. Cells containing both plasmids were then patched onto selective media that lacked histidine, but contained 3-Amino-1,2,4-Triazole (3AT) at the following concentrations 0 mM, 10 mM, 25 mM, 50 mM, 75 mM and 100 mM. After incubation at 30° C. for 24 hours, plates were replica cleaned and incubated at 30° C. for a further 2 days. Growth of MaV203 cells, containing both plasmids, was inhibited in the presence of 3AT at concentrations ≧25 mM. All plates used in the subsequent yeast two-hybrid library screen contained 25 mM 3AT to knockout basal HIS3 expression induced by GAL4 DB-Tanis.

Large Scale Transformation with a Human Liver cDNA Expression Library

MaV203 cells harbouring the pDBB559 plasmid were specially prepared for large-scale transformation with a commercially available cDNA expression library. Specifically 18 μg of plasmid DNA, harvested from a ProQuest™ human liver cDNA library (Life Technologies Inc., USA), was transformed into MaV203 cells containing the pDBB559 plasmid. Approximately 6.0×10⁵ transformants were plated onto selective media containing 25 mM 3AT but lacking leucine, tryptophan and histidine. Transformants that induced the HIS3 reporter gene, and thus contained potential interacting proteins, were selected for further analysis.

Analysis of Reporter Gene Expression

Putative HIS+ positive transformants were streaked for isolated colonies and tested for induction of the associated reporters, URA3 and lacZ. Of the 30 transformants identified as HIS+, only 4 clones, clones 10, 25, 27 and 28 were found to induce at least 2 of the above listed reporter genes.

To further ascertain the authenticity of interaction with Tanis, plasmid DNA from each clone was selectively isolated and re-introduced into MaV203. The re-transformation assay confirmed these clones as containing putative Tanis interacting proteins. All clones were identified as containing plasmids encoding interacting proteins.

Sequence Identification of Positive Clones

Crude plasmid DNA was prepared from the yeast clones 10, 25, 27, and 28, and transformed into DH10B (Life Technologies Inc., USA) electrocompent cells. Cells containing plasmid DNA, encoding the unknown candidate proteins, were isolated from the pool by growth on LB media containing ampicillin at 100 μg/ml. Plasmid DNA for each clone was prepared and partial sequences for the unknown cDNAs determined.

(ii) Results

Bait: Full Length Tanis

Library: Human Liver cDNA Expression Library

Approximately 650 thousand transformants were screened for interactions with Tanis. 4 positive interacting clones were identified and sequenced

Summary of Reporter Gene Expression

| Clone | -HIS | -URA | X-gal | Result |
|---|---|---|---|---|
| Clone 10 | +++ | +ve | Blue | +ve intermediate interacting proteins |
| Clone 25 | +++ | −ve | Blue | +ve weak interacting proteins |
| Clone 27 | +++ | +ve | Blue | +ve weak-intermediate interacting |
| | | (weak) | | proteins |
| Clone 28 | +++ | +ve | Blue | +ve intermediate interacting proteins |

Key-+++ Intermediate growth (scale +-+++++)

Sequences

| | |
|---|---|
| pPC86-Clone 10F | <400>5 |

AAATTNAAACCTTGAAAACCCNACAATGTNTGATGTATATANCTATCTAT
TCGATGATGAANATACCCCACCAAACCCAAAAAAAGAGGGTGGGTCGACC
CACGCGTCCGCCCACGCGTCCGCCCACGCGTCCGCTACAGCACAGATCAG
CACCATGAAGCTTCTCACGGGCCTGGTTTTCTGCTCCTTGGTCCTGGGTG
TCAGCAGCCGAAGCTTCTTTTCGTTCCTTGGCGAGGCTTTTGATGGGGCT
CGGGACATGTGGAGAGCCTACTCTGACATGAGAGAAGCCAATTACATCGG
TCAGACAAATACTTCCATGCTCGGGGGAACTATGATGCTGCCAAAAGGGG
ACCTGGGGGTGCCTGGGCTGCAGAAGTGATCAAGCGATGCCAGAGAGAAT
ATCCAGAGATTCTTTGGCCATGGTGCGGAGGACTCGCTGGCTGATCAGGC
TGNCAATGAATGGGGCAGGAGTGGCAAAAACCCCAATCACTTTCCGACCT
GCTGGCCTGCCTGAGAAATACTGAGCTTNCTCTTNCTCTGTTCTCAAGAG
ATCTGGCTGNGAGGCCTTAAGGCAGGGATACAAAGCGGGGAGAGGGTACA
CAATGGGTATCTAATAAATACTTAAGAAGGNGGGGAANNANNANNNNNNN
NNNNNNANANNAAANGGGCGGCCGNTAANTAAGTAAAACGTCAAACTTTT
AANTAAAGTAAACCGGCCGCCNCCGGGGGGGGAGCTTTTGGACCTTTTTT
CNC

| | |
|---|---|
| pPC86-Clone 25F | <400>6 |

TTNNATTTAACCCTTGTAAATACCCTACAATGGATGATGTATATAACTAT
CTATTCGATGATGAAGATACCCCACCAAACCCAAAAAAAGAGGGTGGGTC
GACCCACGCGTCCGGTTCTGCTACTAGCAACCCTTTTGGTTTAGGTGGCC
TTGGGGGACTTGCAGGTCTGAGTAGCTTGGGTTTGAATACTACCAACTTC
TCTGAACTACAGAGTCAGATGCAGCGACAACTTTTGTCTAACCCTGAAAT
GATGGTCCAGATCATGGAAAATCCCTTTGTTCAGAGCATGCTCTCAAATC
CTGACCTGATGAGACAGTTAATTATGGCCAATCCACAAATGCNGCAGTTG
ATACAGAGAAATCCAGAAATTAGTCATATGTTGAATAATCCAGATATAAT
GAGACAAACGTTGGAACTTGCCAGGAATCCAGCAATGATGCAGGAGATGA
TGAGGAACCAGGACCGAGCTTTGAGCAACCTAGAAAGCATTCCAGGGGGA
TATAATGCTTTAGGCGCATGTACACAGATATTCANGAACCAATGCTGAGT
GCTGCACAAGANCAGTTTGGTGGNAATCCATTTTGCTTTCTTGGTGAGCA
ATACATNCTNTGGTGAANGTAGTCAACCTTCCGTACAGAAAATAGAGATC
ACTACCCAATNCATGGGCTTCCACAGACTTTCCAGAAGTTNATNAAGCTT
TCAACCGGGNACTTGCCAGCACTTGNGGGGGNGGCCTTCTGGGTAGTACT
TGNCNNG

| | |
|---|---|
| pPC86-Clone 27F | <400>7 |

TTTNNATCCNTAAAACCTTGGAAAACCCTACAATGGATGATGTATATAAC
TATCTATTCGATGATGAAGATACCCCACCAAACCCAAAAAAAGAGGGTGG
GTCGACCCACGCGTCCGGCAGCTCAGCTACAGCACAGATCAGCACCATGA
AGCTTCTCACGGGCCTGGTTTTCTGCTCCTTGGTCCTGGGTGTCAGCAGC
CGAAGCTTCTTTTCGTTCCTTGGCGAGGCTTTTGATGGGGCTCGGGACAT
GTGGAGAGCCTACTCTGACATGAGAGAAGCCAATTACATCGGCTCAGACA
AATACTTCCATGCTCGGGGGAACTATGATGCTGCCAAAAGGGGACCTGGG
GGTGCCTGGGCTGCAGAAGTGATCAGCGATGCCAGAGAGAATATCCAGAG
ATTCTTTGGCCATGGTGCGGAGGACTCGCTGGCTGATCAGGCTGCCAATG
AATGGGGCAGGAGTGGCAAAGACCCCAATCACTTCCGACCTGCTGGCCTG
CCTGAGAAATACTGAGCTTCCTCTTCACTCTGCTCTCAGGAGATCTGGCT
GTGANGCCCTCANGGCANGGATACAAAGCGGGGAGAGGGTACACAATGGG
TATCTAATAAATACTTAAAGANGNGGGAAANANNNNNNNNNNNNNNNNNN
NNNNNNNNNNNANAAAAAAANNNGGGGGCGGGCCGTTAAGTAAAGNAANA
ACGTTNNACTTTTAANTNAAGTAACCGGGCNGCCNCCGGGGGGGGGAGCT
TTGGGACTTTTTTC

| | |
|---|---|
| pPC8G-Clone 28F | <400>8 |

TNNNATNAGNATACNCTTTGAAAAACCANGACAATGGATGATGTATATAA
CTATCTATTCGATGATGAAGATACCCCACCAAACCCAAAAAAAGAGGGTG
GGTCGACCCACGCGTCCGCCCACGCGTCCGCAGCTACAGCACAGATCAGC
ACCATGAAGCTTCTCACGGGCCTGGTTTTCTGCTCCTTGGTCCTGGGTGT
CAGCAGCCGAAGCTTCTTTTCGTTCCTTGGCGAGGCTTTTGATGGGGCTC
GGGACATGTGGAGAGCCTACTCTGACATGAGAGAAGCCAATTACATCGGC
TCAGACAAATACTTCCATGCTCGGGGGAACTATGATGCTGCCAAAAGGGG
ACCTGGGGGTGCCTGGGCTGCAGAAGTGATCAGCGATGCCAGAGAGAATA
TCCAGAGATTCTTTGGCCATGGTGCGGAGGACTCGCTGGCTGATCAGGCT
GCCAATGAATGGGGCAGGAGTGGCAAAAGACCCCAATCACTTNCGACCTG
CTGGCCTGGCTGAGAAATACTGAGCTTTCTCTTNCTCTGCTCTCAAGAGA
TCTGGCTGTGAGGCCCTCAGGGCAGGGATCAAAAGCGGGGAGAGGGTACA
CAATGGGTATCTAATAAATACTTAAGANGNGGGAAAAAAAAAAANNANNN
NNNNNNNNNNNNNNNNNNNNNNNNNNNTNNNNTNNNNNNNNNNNNNNNNN
NNNANNAAANAAANNNNGGGGGNGGCCCTTTAANAAAANNAAAAACGNCA
ACCT

| | |
|---|---|
| pPC8G-Clone 10R | <400>9 |

TCCGAAAACCCTTTTGAANACGCCCNAGAGACATTNACCAAACNTCTGGC
TGATAGAAGTCCAAAGCTNCACCGCGGTGGCGGNCGNTACTTNTTTAGAG
CTCGACGTCTTACTTACTTAGCGGCCGCCCTTTATTTTTTTTTTTTTTTT
TTTTTTTTTTCCCNCCTNTTAAGTATTTATTANATACCCATTGGGTACCC
TNTCCCCGNTTTGTATCCCTGCCCTGNGGGCCTNACAGCCAGATNTCCTG
ANAGCANAGNGAAAAGGAAGCTCATTTTTTTTCAGGCAGGCCACCAGGTC
GGAAGNGATTGGGGTNTTTGCCNCTCCTGCCCCATTCATTGGCAAGCCTG
ATCAGCCAGGGNGTNCTCCCCNCCATGGCCAAAAAANTTTTGGANNTTNT
TTNTGGCATCGGTGATNACTTTTGNAGCCCAGGCCCCCCCAGGNCCCCTT
TTGGGANCATTATAGTTCCCCCGNGCATGGAAAGTNTTTGCTGACCGATG
NAATTGGCTTNTTTNATTGTAAAAAAANGCTNTTCANATGTCCNAGCCCA
TNAAAAGCTTTGCCAAAGGAACAAAAANAAACNTTTGGNTGGTGACCCCC
CAGGGACCCAAGGGGCAANAAAACCNGGCCCCGNGAAAAAGCTTNATNGG
GGCTTGAANTGGNGCTGGAACCGGGAACCCCTTGGGCCGGCCCCNTNGGG
CCGGCCCCTTGGGGTCAACCCCCCCCTTTTTTTTTTNGGGGTTNGGGGGG
GGGGATTNTTCATANNNCNNAAAANA

| | |
|---|---|
| pPC86-Clone 25R | <400>10 |

AGCCNCAACCTTGATTGGAGACTTGACCAAACCTCTGGCGAAGAAGTCCA
AAGCTCCACCGCGGTGGCGGCCGTTACTTACTTAGAGCTCGACGTCTTAC
TTACTTAGCGGCCGCCCTTTTTTTTTTTTTTTGCTTCTTTTTAATGCTTT
TATTCTACATAAATTACTACCATAGGCTAATGTTTAAAAAGCAAATAAAC
TGGACAGATGCAGGACAAAATCTGGTCACCCAACTATAAAAGGTGATGTT
TTTAAAAAATTACAATAAATGCAGAAGTGATGCATGCAGTAGCCTTAATT
CCCACTGTTCCAGAAAAGAAAAATACAGAAAAACCCACACATCTTACTGT
ACTCCACCTTAAAATGCATCATATTGGGTTTGTTTATAACAGCACAGAAT
TCCAAGAGTCAAAATGAAATAAAGCAGGTATTTTAAAGTTTAAGAGCCGT
TATCAAAAATAAATTACATTTTTTCAAGATACAGAAATGCTGCTATGATG
GCTGGGAGCCCAGTAACCTTTTCAATAGCTGCATTGATATCACTNCTGGT
TGCTATTAGAGCTTGCAAGGTTGCTTTCACCGGNTCAAAAAATNCCANTG
GCACTTGAGTTGGTTNCANGNTGGTGGCTGNAAATCTGGACTTNTTGGGA
TTTNTGGAGGCTGGNGGGATTTACCTNCAACCAAGGAACCCTGGAANCCA
TTNTGGCTGGAANNAACCTGGCTGGATGTTCCAAGGTTCAAGGGGGTTNC
CGNGTGGGGGGGACCTTGGGNNTTTCACTAAGGGGGGGGGGGTTAGNANN
CCATTTAGTTTCCCCGAAAAAGCCTCCAANGGNTTTCTAANGGNCCCCCA
AACCANGANATAAANCCTGGGATGAAGGNCCCGGGCTTTCNTNTNT

| | |
|---|---|
| pPC86-Clone 27R | <400>11 |

TTNAATCAAGCCGACAACCTTGATTGGAGACTTGACCAAACCTCTGGCGA
AGAAGTCCAAAGCTCCACCGCGGTGGCGGCCGTTACTTACTTAGAGCTCG
ACGTCTTACTTACTTAGCGGCCGCCCTTTTTTTTTTTTTTTTTTTTTTTT
TTTTTTTTTTTTTTTTTTTTTTNCCNCCTNTTAAGTNTTTATTANANNCC
CATNGGGTNCCCTNTCCCCGNTTTGTATCCCTGCCCTGNGGGCCTNANAG
CCANATNTCCTGANAGCANAGNGAAAAGGAANCTCANTNTTTNTNAGGCA
GGCCANCAGGTCGGAAGGGATNGGGGTTTTTGCCACTCCTGCCCCATTCA
TTGGCAGCCTGATCAGCCAGNGAGTCCTCCGCNCCATGGCCAAANAATCT
NTGGATNTTNTTTTTGGCATCGTTGATCACTTTTGCAGCCCAGGCNCCCC
CAGGTCCCCTTTTGGCAGNATAATAGTTCCCCCGAGCATGGAANTATTTN
TCTGACCCNANGTAAATTGGGCTTNNTTTATGTCAANAGAGGGCTTTCAC
ATGTCCCGAGCCCCCATNAAAAAAGCCTCGCCAAGGGNACNAAAANAANC
TTTTGGNTGGTGGCNCCCCGGNACCNAGGGGGCAAAAAACCCGCCCCCGN
GAAAAANCTTNAATGGGGCNTNATCTTGGGCTNGAACCTTNAACCGNCCG
GACCCCCTNNGGTTCCANCCCCCCCTTTTTTTTTTNGGGGTTNGGGGGGG
GGGTTTTTTTCNTCACCGGAAANANAAAGGGNTTTATATANCCCCNNCCC
TTGGGGGGGGGGGATTAAAAAACNCCCCNGGGGGGGATTNCCAAACCCCN
TTTNANCCCANGTTNGGNNCCCCCCCNCCCGGGGGACANGGGGTTTTNNA
ATANAAC

| | |
|---|---|
| pPC86-Clone 28R | <400>12 |

TTNATNNAATCAAGCCGACAACCTTGATTGGAGACTTGACCAAACCTCTG
GCGAAGAAGTCCAAAGCTCCACCGCGGTGGCGGCCGTTACTTACTTAGAG
CTCGACGTCTTACTTACTTAGCGGCCGCCCTTTTTTTTTTTTTTTTTTTT
TTTTTTTTTTTTTTTTTTTTTTTTTTTTTTTTTTTTTTTTTTTTTTTTTT
TTTTTTTTTTTTTTTTNCCCCCCTTTAAANNNTTAANNAAAACCCCNTNG
GGCCCCCNNNCCCNNTTTTNTNNCCNNNCCNGGGGGGCCNAAAANCCANN
TTTNNNGAAANNANNGGNAANNGNAANNNNTTTTTTTTTAAGNCNNCCCA
NNAGGNNGAAAGGNATGGGGGTTTTTNCCNCNCCNNCCCNNTTNNTNGGC
ANCNTNAAAANCCAGGGNGNCCNCCCCNCNANGGCAAAAAAATTTNGGNA
TTTTTTTTNGGGNANGGNGGAAAAATTTNGGACCCCAAAGCCCCCCNAGG
NCCCNTTTTGGAAAAAAAAANTTTCCCCCCNAAAAAGGAAAANTTTTTTT
CNNANAACCCAAAAAAAAANTNNCNTTTTTTTAAAGGGGAAANNAAAGGG
NTTTNCCCNAAATNTTCCCNNCCCCCNANAAAAAAAACCCNCTCCNAAAA
AAAAAAAAAAAAAAAAATTTTTGNGTTTGTTNGCCCCCCCGGNCCCANGG
GGGGGNAAAAAACCCCCCCCCCCGGNAAAAAACNTTAANGGGGGCCANAN
TTTTGGCNTAAAAATCCGNACCCCCNNGGGGGCAACCCCCTNNGTCCCAN
CCCCCCTTTTTTTTTNGGGGNGGGGGGGGGGTTTTCNNCNNCGAAAANGG
GGGGTNAACCCCCCCCCNGGGGGGGGGNNAAAANCCCCCGNNGGAANCCN
ANCCC

Clone Identity:
Clone 10: Serum amyloid A (SAA)
Clone 25: Ubiquilin
Clone 27: SAA
Clone 28: SAA
   Summary of Sequence Analysis:

Clones 10, 27 and 28: 97% homology to Homo sapiens serum amyloid A (SAA) mRNA, complete code. (Genbank accession: M26152)

Serum amyloid A (SAA) proteins comprise a family of proteins that associate predominantly with HDL and SAA is considered an acute phase response protein as its synthesis is greatly increased (up to as much as 1000 fold) in inflammation. Increased acute phase response proteins are detected in type 2 diabetics, including SAA. Increased SAA in type 2 diabetes may act to redirect HDL cholesterol from the liver to the macrophage for tissue repair. This increased catabolism is thought to be a possible reason for the low HDL concentrations observed in diabetic patients, and the uptake by macrophages in the atherosclerotic plaque could be part of the reason for an increased risk of arterial disease in type 2 diabetics.

Clone 25: 97% homology to human PLIC-1 mRNA (also known as human ubiquilin 1). PLIC-1 (proteins linking integrin-associated protein and cytoskeleton), also called ubiquilin, physically associates with both proteosomes and ubiquitin ligases in large complexes. When over-expressed, hPLIC proteins interfere with the in vivo degradation of two unrelated ubiquitin-dependent proteasome substrates, p53 and IkappaBalpha, but not a ubiquitin-independent substrate. Recent studies suggest that the hPLIC proteins, and possibly related ubiquitin-like family members, may functionally link the ubiquitination machinery to the proteasome to affect in vivo protein degradation.

### EXAMPLE 2

### Biacore Confirmation of Tanis Interaction with SAA

Biacore J is a general-purpose system for monitoring the presence and properties of biomolecules, based on affinity biosensor technology. The system works by detecting binding between specific pairs of molecules, where one binding partner is attached to a sensor surface and the other is present in sample solution passed over the surface. Biacore J is capable of detecting binding partners, by monitoring whether a response is obtained when samples are passed over the sensor surface. It can also monitor and compare binding activities, by examining the shape of the binding curve. Concentrations of analytes (sample in solution) by measuring the response obtained and comparing with a standard curve obtained from known samples.

The detection principle used in biacore J does not require labelling of the molecules being investigated, and applies to any kind of biomolecule including proteins, nucleic acids, carbohydrates, lipids and conjugate molecules.

The output generated by biacore J is a curve, obtained by plotting response against time, this is termed the sensorgram. Illustrated below is a typical sensorgram showing both negative and positive binding results. Characteristics of the curve, when analysed can provide details as to the nature of the binding event.

(i) Materials and Methods

Recombinant Tanis Production

The Tanis gene has been cloned into the pGEX5X1 vector (Amersham Pharmacia Biotech AB, Sweden). Plasmids have been constructed to express the Tanis protein with an N-terminal GST fusion protein tag. In total four constructs have been produced, expressing either full-length Tanis or one of the three fragments cloned, N-term (1-37aa), Cplus (53-189aa) or C (117-189aa). The DNA sequence of each of these plasmids has been confirmed. Each plasmid has been introduced separately into the BL21 strain of E. coli (Amersham Pharmacia Biotech AB, Sweden) this strain contains the gene for the bacteriophage T7 RNA polymerase which is specific for the T7 promoter contained in the pGEX5X1 plasmids. This polymerase gene is under the direction of the LacZ promoter which is induced by the addition of isopropothiogalactosidase to a log phase BL21 bacterial culture. Post induction the cells are harvested by centrifugation and the cell pellet resuspended in a cell lysis buffer. The cells are then lysed using ultrasonics generated by a digital sonifier (Branson, USA). Soluble and insoluble proteins are separated by centrifugation with the supernatant containing the soluble proteins, including the GST-tanis. Only the soluble fraction is processed, the insoluble fraction is discarded. Glutathione sepharose (Amersham Pharmacia Biotech AB, Sweden) has a high affinity for the GST protein. GST-tanis protein is bound to the resin as the recovered soluble fraction is passed across. Washing of the resin removes other contaminating proteins. The extracted GST-tanis protein is recovered by the addition of 10 mM reduced glutathione (Amersham Pharmacia Biotech AB, Sweden) to the resin bed. The degree to which the GST-tanis protein has been purified is determined by SDS-PAGE analysis.

A control GST alone protein was also produced using the same method outlined above.

Full length Tanis (FL-tanis) amino acid sequence
(189aa) <400>13
MESAEEPLPARPALETEGLRFLHVTVGSLLASYGWYVLFSCILLYIVIQK
LSVRLRVLRQRQLDQADAVLEPDAVVKRQEALAAARLRMQEDLNAQVEKH
KEKLRQLEEEKRRQKIEMWDSMQEGRSYRRNPGRPQEEDGPGPSTSSSVT
RKGKSDKKPLRGNGYNPLTGEGGGTCAWRPGRRGPSSGG
N-terminal tanis (1-37aa) <400>14
MESAEEPLPARPALETEGLRFLHVTVGSLLASYGWYV
C-plus tanis (53-189aa) <400>15
VRLRVLRQRQLDQADAVLEPDAVVKRQEALAAARLRMQEDLNAQVEKHKE
KLRQLEEEKRRQKIEMWDSMQEGRSYRRNPGRPQEEDGPGPSTSSSVTRK
GKSDKKPLRGNGYNPLTGEGGGTCAWRPGRRGPSSGG
C tanis (117-189aa) <400>16
EMWDSMQEGRSYRRNPGRPQEEDGPGPSTSSSVTRKGKSDKKPLRGNGYN
PLTGEGGGTCAWRPGRRGPSSGG

Serum Amyloid A (SAA) Protein

A 96% pure sample of SAA purified by size exclusion chromatography from human sera (Trace Scientific, Australia) was used for all experiments.

Tanis-SAA Interaction Study Using Biacore J

The 96% pure SAA was covalently bound to the biospecific sensor surface of the Biacore J (biacore, Sweden). This surface consists of a gold-coated glass slide mounted in a plastic holder (the sensor chip). The CM5 chip (biacore, Sweden) was used for all experiments and this chip is covered with a hydrophilic matrix, consisting of carboxymethylated dextran to which the SAA was covalently bound. The tanis protein fragments were injected into the system and flowed across this chip and sensorgrams were recorded in real-time.

(ii) Results

Reference point 1 is the base line each injection of GST-FLtanis has subsequently increased the resonance units (RU). The interaction of the proteins is indicated by the failure of the response to return to the baseline RU value. The difference between the values post injection (points 1, 3, 5, 7) indicates a binding event between full length tanis and SAA. The difference between the injection point and the max RU indicates the affinity between the two proteins. The gradual decline in RU after the maximum point shows the dissociation of the binding event. These sensorgrams indicate a potentially high affinity binding with a rapid dissociation of the binding event. A series of controls have also been injected across the SAA chip, these were GST alone, BSA (NEB, USA) alone and buffer alone. None of these produced a response similar to that shown above, indicating that this is a true response.

GST fused fragments of tanis have been tested to potentially determine the region of the tanis protein which is interacting with SAA (shown below). The N-terminal, C-terminal and C-terminal plus coiled coil regions have each been injected across the SAA chip. The chemically synthesised 24aa peptide (N-terminal) was also injected. Both forms of the N-terminal tanis produced no change in RU value, yielding no response. Both the Cplus and C regions produced a curve similar to that seen with the FLtanis. The result achieved with the Cplus region has since been reproduced with a different batch of protein suggesting that there is an interaction occurring. The result from the Cplus and C experiments suggest that the region of the tanis protein interacting with SAA is located within the C-terminal 72 amino acids.

### EXAMPLE 3

### A Link Between Type 2 Diabetes and Inflammation

(i) Research Design and Methods

Experimental Animals.

A colony of P. obesus is maintained at Deakin University (Geelong, Australia). Breeding pairs are fed a diet of lucerne and standard diet ad libitum. Experimental animals were weaned at 4 weeks of age and given a standard laboratory diet, from which 12% of energy was derived from fat, 63% from carbohydrate, and 25% from protein (Barastoc, Pakenham, Australia). Animals were housed in a temperature-controlled room (22±1° C.) with a 12-h light-dark cycle (light 06:00-18:00). Animals were classified as normal glucose tolerant (nGT), impaired glucose tolerant (IGT), or type 2 diabetic at 16 weeks of age according to their blood glucose and plasma insulin concentrations as previously described. Whole blood glucose was measured using an enzymatic glucose analyzer (Model 27, Yellow Springs Instruments, Yellow Springs, Ohio). Plasma insulin concentrations were determined using a double-antibody solid-phase radioimmunoassay (Phadeseph, Kabi Pharmacia Diagnostics, Sweden).

Differential Display PCR.

At 18 weeks of age, animals (n 6 from each group; nGT, IGT, and type 2 diabetic) were randomly assigned and either fasted for 24 h or fed ad libitum. After 24 h, the animals were killed and the tissues were immediately removed and frozen in liquid nitrogen. RNA was extracted from tissues using RNAzol B (Tel-Test, Friendswood, Tex.) and reverse-transcribed using Superscript II (Invitrogen Life Technologies, Rockville, Md.). Differential display PCR was performed on liver cDNA using an RNAimage mRNA Differential Display System (GenHunter, Nashville, Tenn.). The Tanis gene was identified using the G anchored primer (5′-aag ctt ttt ttt ttg-3′) (SEQ ID NO:30) and an arbitrary primer (5′-aag ctt ctc aac g-3′) (SEQ ID NO:31).

Sequencing.

DNA sequencing was performed using the ABI PRISM BigDye Terminator Cycle Sequencing Ready Reaction Kit and a 373 automated fluorescent DNA sequencer (PE Applied Biosystems). The 5′ and 3′ ends of the transcript were determined by RACE of a Marathon cDNA Library (Clontech, Palo Alto, Calif.).

Measurement of Tanis Gene Expression.

The level of Tanis gene expression in each cDNA sample was quantified using Taqman PCR technology on an ABI Prism 7700 sequence detector. β-Actin was used as an internal standard to normalize the amount of cDNA in a reaction. Primer sequences were as follows: Tanis gene forward, 5′-gat gcg ttc aat gat gtc ttc ct-3′ (<400>22); Tanis gene reverse, 5′ ga agc aaa ccc cat caa ctg t-3′ (<400>23); β-actin forward, 5′-gca aag acc tgt atg cca aca c-3′ (<400>24); β-actin reverse, 5′-gcc aga gca gtg atc tct ttc tg-3′ (<400>25). Fluorogenic probe sequences were 5′-cac atc agt aat cct cac tgg tgg gct ac-3′ (<400>26) for the Tanis gene and 5′-tgc tgg cac cag act tgc cct c-3′ (<400>27) for the β-actin gene. The Tanis and β-actin probes had the reporter dyes FAM and VIC, respectively, attached to the 5′-end, and both probes had the quencher dye TAMRA attached to the 3′ end. PCR conditions were 50° C. for 2 min and 95° C. for 10 min followed by 40 cycles of 95° C. for 15 s and 60° C. for 1 min.

Cell Culture.

Tanis gene expression was studied in three cell lines: HepG2 hepatocytes (European Collection of Cell Cultures), C2C12 myotubes (American Type Culture Collection), and 3T3-L1 adipocytes (a gift supplied by Dr. Lance Macaulay, CSIRO, Parkville, Australia). All cells were routinely cultured in Dulbecco's modified Eagle's medium (5-25 mmol/l glucose), 10% fetal calf serum, and antibiotics (Life Technologies, Melbourne, Australia).

Yeast-2 Hybrid Screen.

Yeast-2 hybrid screening was performed using the ProQuest Two Hybrid System (Life Technologies). The coding sequence of Tanis was cloned into the yeast vector pDBLeu and transformed into DH5a cells by electroporation. Recombinant clones were detected by PCR using vector-specific primers (forward 5′-gaa taa gtg cga cat cat cat c-3′ (<400>28); reverse 5′-gta aat ttc tgg caa ggt aga c-3′ (<400>29)). One positive clone, pDBB559, was selected for use in yeast-2 hybrid screening. The sequence of the insert was confirmed to be 100% homologous to the Tanis cDNA sequence and cloned in frame with the GAL4 DNA binding domain of pDBLeu. PDBB559 was transformed into the yeast strain MaV203, and the amount of 3-Amino-1,2,4-Triazole (3AT) required for suppression of basal HIS3 expression of the transformants was determined by titration of cell growth on plates containing varying amounts of 3AT (0-100 mmol/l). MaV203 cell growth was inhibited at 3AT concentrations over 10 mmol/l, and all plates used in the subsequent yeast-2 hybrid library screen contained 25 mmol/13AT to suppress basal HIS3 expression induced by GAL4 DB-Tanis. MaV203 cells harbouring the pDBB559 plasmid were specially prepared for large-scale transformation with a commercially available cDNA expression library. Specifically 18 μg of plasmid DNA, harvested from a ProQuest human liver cDNA library, was transformed into MaV203 cells containing the pDBB559 plasmid. Approximately 6.0×10⁵ transformants were plated onto selective media containing 25 mmol/13AT but lacking leucine, tryptophan, and histidine. Transformants that induced the HIS3 reporter gene were predicted to contain potential interacting proteins and were selected for additional analysis. Putative HIS-positive transformants were tested for induction of two other associated reporters, URA3 and lacZ. Of the transformants initially identified as HIS+, four clones also were found to be positive in inducing expression of URA3 and lacZ. The plasmids isolated from these four clones were then sequenced using standard methods.

Expression and Purification of Recombinant Tanis and Serum Amyloid A Proteins.

The cDNA encoding the complete 189 amino acid Tanis protein, the cDNA corresponding to the region coding for a COOH-terminal (amino acids 54-189, termed Tanis-C) fragment of Tanis and cDNA encoding the mature sequence of serum amyloid A (SAA) 1β (amino acids 19-122) were ligated into the pGEX-5X-1 expression vector (Amersham Pharmacia Biotech, Buckinghamshire, U.K.). The GST, GST-full-length Tanis, GST-Tanis-C, and GST-SAA proteins were expressed in the B121 strain of Escherichia coli and affinity-purified using Glutathione Sepharose beads (Amersham Pharmacia Biotech). The quality and quantity of the expressed proteins were checked by SDS-PAGE and Coomassie blue staining. The GST, GST-Tanis-C, and GST-SAA expressed and purified well, whereas GST-full-length Tanis showed weak expression and only a small amount of protein was recovered on purification. GST protein served as a control in all experiments involving recombinant GST fusion proteins.

Surface Plasmon Resonance Analysis.

The real-time protein-protein interactions were examined by surface plasmon resonance (SPR) analysis using a Biacore J instrument purchased from Biacore AB (Uppsala, Sweden). The system detects binding between specific pairs of molecules where one (ligand) is attached to the surface of a sensor chip and the other (analyte) present in a sample solution is passed over the surface. HBS buffer (10 mmol/l HEPES [pH 7.4], 150 mmol/l NaCl, 3 mmol/l EDTA, 0.005% polysorbate 20) was used as running buffer in all SPR experiments. SAA, purified from human plasma (95% pure), was purchased from Trace Scientific (Australia). The SAA or GST-Tanis-C or GST-SAA was immobilized covalently to a sensor chip (CM5) via amine coupling by carbodiimide chemistry using the reagents supplied (Amine coupling kit, Biacore AB). Preconcentration tests were performed and found pH 4.0 to be most suitable for coupling ligands to CM5 chips. To test for interaction, the analyte samples were diluted (GST, GST-Tanis, GST-Tanis-C, or GST-SAA) into running buffer and injected them into the system when the sensorgram exhibited a stable baseline with noise levels <2 resonance units (RU). The chips were regenerated between uses by injection of 10 mmol/l Glycine-HCl (pH 2.0) for 4 min. Statistical analysis.

All data are expressed as mean ±SE. Comparisons between groups were made by analysis of variance with post hoc least-significance difference tests. Differences were considered significant at P<0.05.

(ii) Results

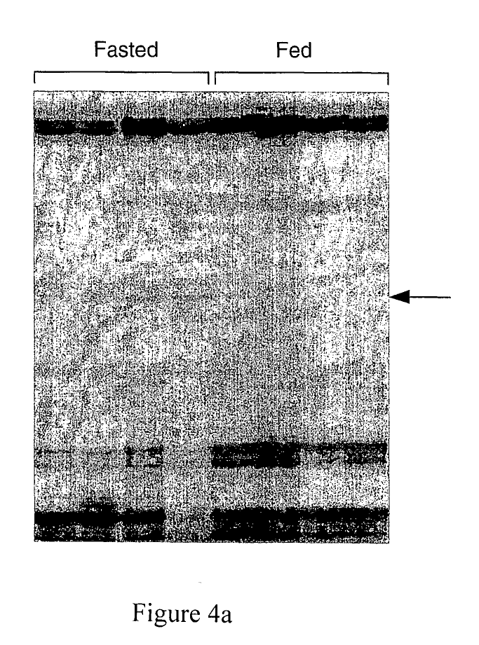

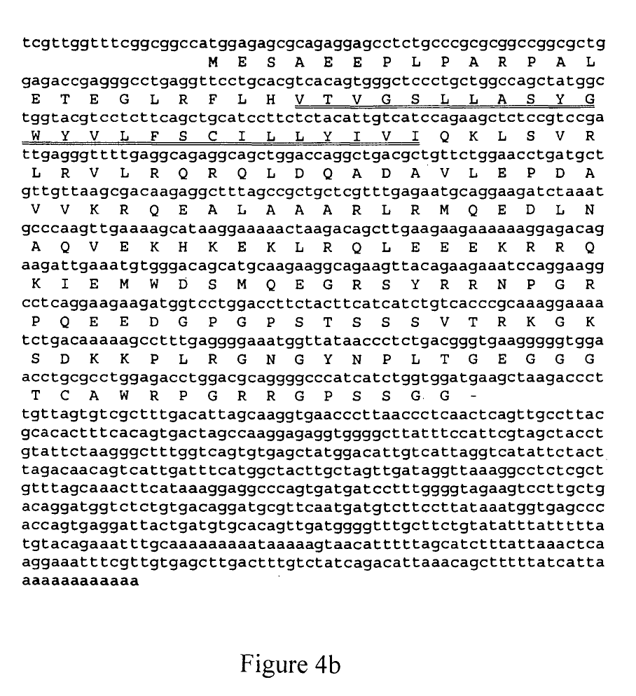

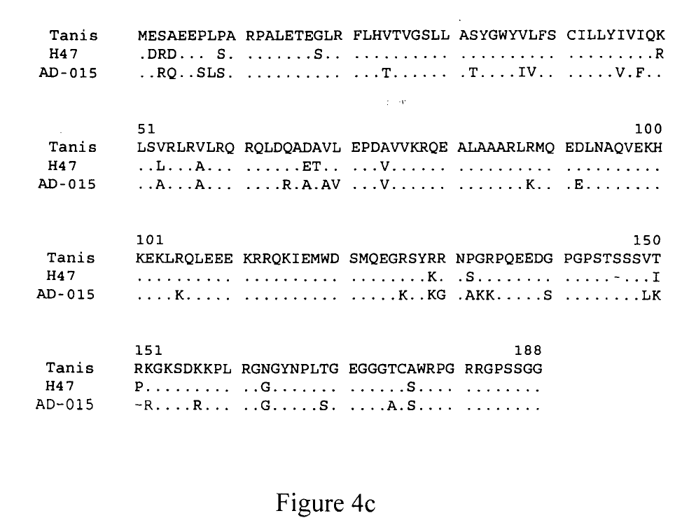

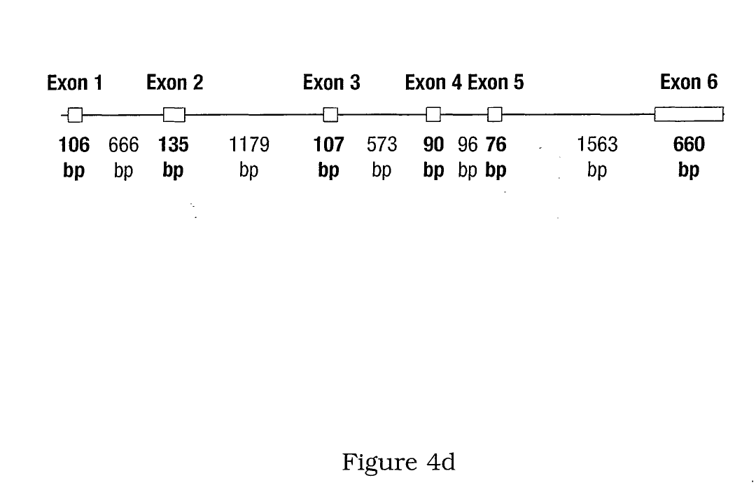

Differential display PCR was used to identify a gene whose expression was elevated in the liver of fasted P. obesus relative to fed controls (FIG. 4A). This band was excised and sequenced, revealing it to be a novel P. obesus gene with no apparent homologues in the public databases. This gene was named “Tanis”, a Hebrew word for fasting. Subsequently, several apparently homologous sequences have been submitted to Genbank (e.g., accession no. AF157317, AF335543). The entire P. obesus Tanis mRNA sequence, obtained using RACE, is shown in FIG. 4B. The predicted Tanis amino acid sequence is given in FIG. 4C, along with alignments to apparently homologous genes from other species. A high level of identity was evident between species (Table 4), indicative of a conserved gene with important physiological function.

TABLE 4
Conservation (% identity) of Tanis at the nucleotide and amino
acid levels in various species.

| | Nucleotide | | | | Amino acid | | | |
|---|---|---|---|---|---|---|---|---|
| | Po. | Hs. | Rr. | Mm. | Po. | Hs. | Rr. | Mm. |
| P. obesus (Tanis) | — | 84 | 93 | 90 | — | 80 | 91 | 90 |
| H. sapiens (AD-015) | 84 | — | 85 | 84 | 80 | — | 86 | 81 |
| R. rattus (EST) | 93 | 85 | — | 96 | 91 | 86 | — | 96 |
| M. musculus (H47) | 90 | 84 | 96 | — | 90 | 81 | 96 | — |

Accession numbers: P. obesus (Tanis; H. sapiens (AD-015), XM007631; R. rattus, AA893841; M. musculus (H47), AF335543.

P. obesus mRNA for Tanis consists of 1,155 nucleotides and encodes a protein of 189 amino acids. Sequence analysis of the predicted Tanis protein suggested a single transmembrane region (amino acids 26-48), a dileucine motif, and a coiled-coil region.

Analysis using Expasy software tools (http://www.expasy.ch) predicted eight possible serine phosphorylation sites, one threonine phosphorylation site, three possible O-glycosylation sites, and four possible protein kinase C phosphorylation sites). Tanis could not be assigned to any known gene families and was predicted to have an overall composition of 44% a helix, 17% extended sheet, and 39% random coil.

The genomic structure of the P. obesus Tanis gene was determined by direct sequencing of gDNA and cDNA samples, and is shown in FIG. 4D. The gene consists of six exons ranging in size from 76 to 660 nucleotides. Exon 6 includes coding sequence for the COOH-terminal 25 amino acids and 585 nucleotides of 3′ untranslated region. The corresponding human gene, known as AD-015, was derived by automated computational analysis of genomic sequence at the National center for Biotechnology Information (NIH, Bethesda, Md.) using gene prediction. The contig containing this sequence was localized to human chromosome 15q26.3 in the interval D15S157-qTEL, with the nearest marker identified as stSG26005. Of interest is that the syntenic chromosomal region in mice and pigs contains four obesity-related QTL: Qw7 (19), Bw61 (20), Pfat1 (21), and SSC7 (22), suggesting that a gene in this region affects body fat accumulation.

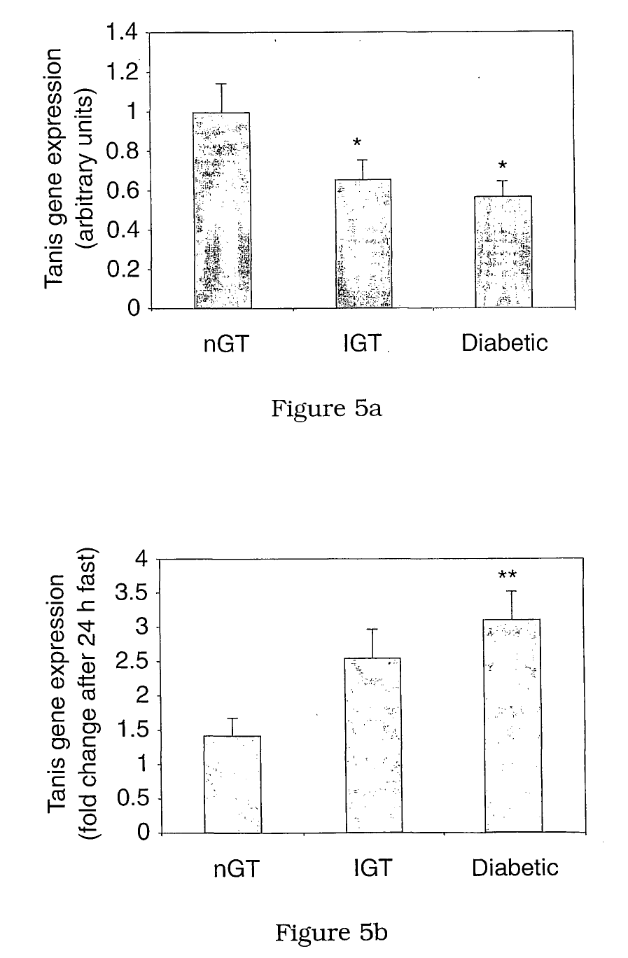

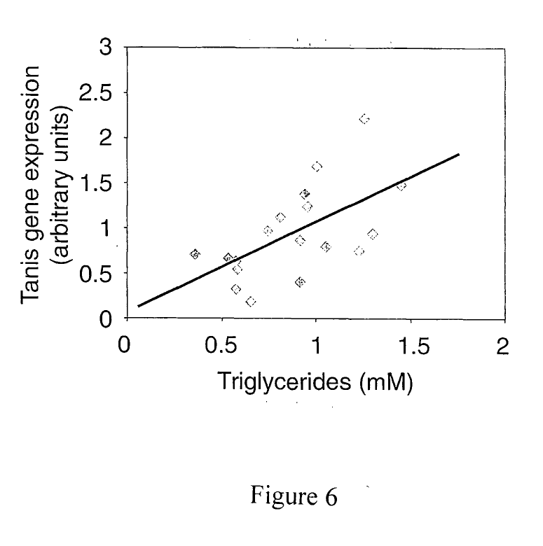

Hepatic Tanis gene expression was increased 2.2-fold after a 24-h fast in P. obesus (P<0.001). Within the subgroups of animals, the increase in hepatic gene expression of Tanis after fasting was significant only in the diabetic animals (3.1-fold increase; P=0.010; FIG. 5). In ad libitum-fed animals, expression of the Tanis gene in the liver was reduced in both IGT (P=0.039) and type 2 diabetic P. obesus (P=0.015) relative to their nGT littermates (FIG. 5). In addition, linear correlations were observed between Tanis expression and circulating triglyceride concentrations (Pearson r=0.593, P=0.007; FIG. 6), as well as blood glucose (Spearman r=−0.378, P=0.010) and insulin concentrations (Spearman r=−0.416, P=0.004) in ad libitum-fed P. obesus. There was also evidence of a correlation between hepatic Tanis gene expression and the change in blood glucose (Spearman r=0.395, P=0.010) and insulin concentrations (Pearson r=0.374, P=0.015) after 24 h of fasting. Multiple linear regression analysis indicated that only the change in blood glucose concentration was independently associated with Tanis gene expression (P=0.004), suggesting a relationship between these two variables. In addition, when dietary energy restriction was continued for a period of 2 weeks (at 67% of normal dietary intake), Tanis gene expression in the liver was increases 2.2-fold relative to ad libitum-fed control animals (1.91±0.29 vs. 0.87±0.08 arbitrary units, P=0.006).

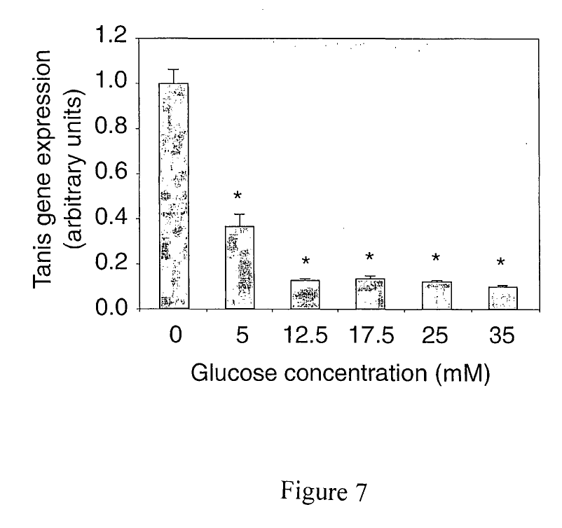

In accordance with the results obtained in vivo, cell culture experiments showed that Tanis gene expression in HepG2 hepatocytes was profoundly regulated by media glucose concentration (P=0.006; FIG. 7). Increasing the concentration of glucose in the media caused a dose-dependent reduction in the levels of Tanis gene expression, with a maximal effect observed at 12.5 mmol/l glucose of 90% suppression.

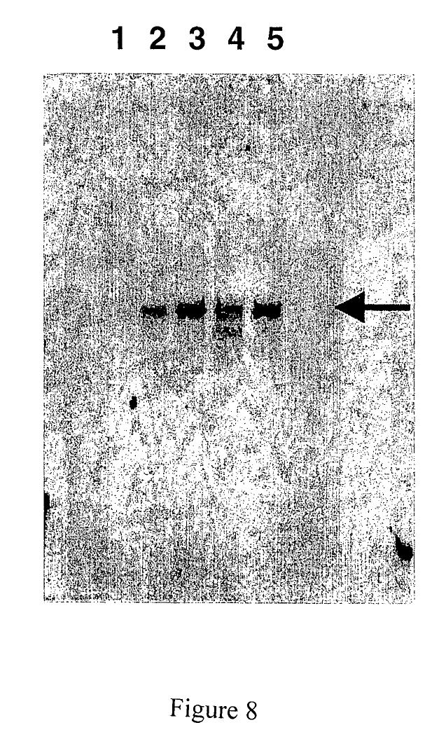

Tanis gene expression was tested in tissues other than liver using both Taqman PCR and Northern blots. Tanis gene expression was detected by Taqman PCR in all tissues examined, including hypothalamus, liver, skeletal muscle, adipose tissue, testes, heart, and kidney. Northern blotting revealed a single band of the expected size (1,155 nt) in a range of tissues, including liver, adipose tissue, hypothalamus, and skeletal muscle (FIG. 8).

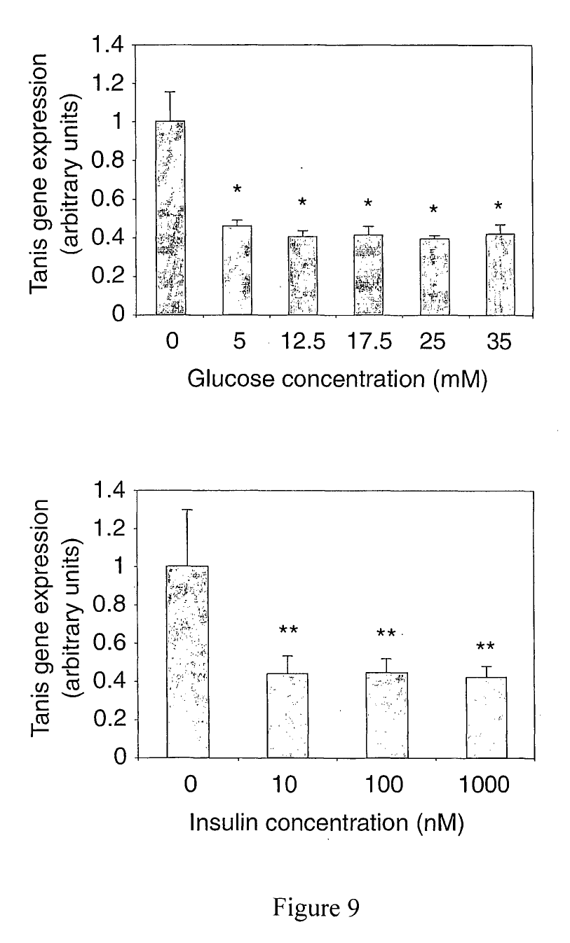

Expression profiling of the Tanis gene in nonhepatic tissues revealed no effect of fasting on Tanis gene expression in adipose tissue, muscle, or hypothalamus (data not shown). However, in vitro Tanis gene expression was suppressed in a dose-dependent manner by glucose in 3T3-L1 adipocytes (FIG. 9) and C2C12 muscle cells (maximum effect of 50% suppression at 25 mmol/l glucose; P=0.002). Tanis gene expression was also decreased by insulin in 3T3-L1 cells (FIG. 9) and in C2C12 cells.

To examine further the physiological role of the Tanis protein, a yeast-2 hybrid screen was conducted to identify interacting proteins. Using Tanis as bait, ˜600,000 transformants from a human liver cDNA library were screened. Expression analysis of three different reporter genes independently confirmed four clones to be positive, with each showing evidence of interaction of intermediate strength. Sequencing of these clones revealed that three of the four encoded SAA, an acute-phase inflammatory response protein. The entire nucleotide sequence of all three positive clones identified in the yeast-2 hybrid screen matched with the known sequence of human SAA1β, an allele of the SAA1 gene (Genbank accession no. CAA39974).

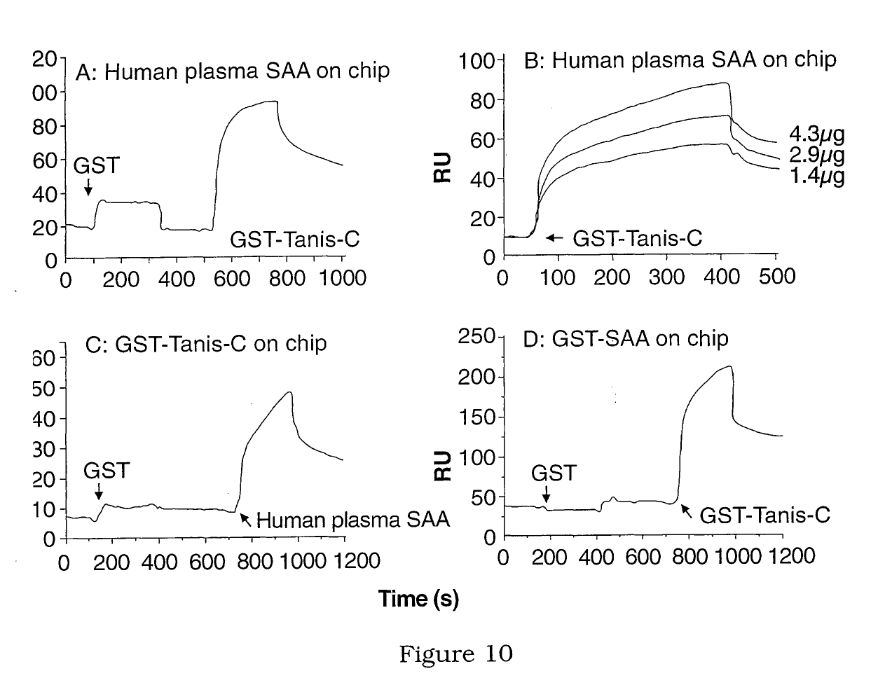

The putative interaction between Tanis and SAA was confirmed by SPR analysis. A CM5 sensor chip with 4,737 RU of human plasma SAA coupled to its surface was initially used for testing interaction with GST-full-length Tanis. The sensorgram revealed a binding phenomenon with GST-Tanis. GST-full-length Tanis, which contains the predicted transmembrane domain, was difficult to express and purify to a satisfactory degree. Therefore, the COOH-terminal 136 amino acid fragment of Tanis, which does not include the transmembrane region, was expressed and purified. The GST-Tanis-C protein was expressed and purified at satisfactory levels. In the SPR binding analysis, GST-Tanis-C demonstrated binding with human plasma SAA, which was concentration-dependent (FIGS. 10A and B). Interaction was shown to be positive even in the reverse situation where GST-Tanis was bound to the sensor chip as a ligand and SAA was passed through as an analyte (FIG. 10C).

SAA purified from human plasma is a heterogeneous sample likely to contain all of the forms of SAA present in circulating blood. To examine interactions with a homogeneous sample, SAA1β was expressed and purified as a GST fusion protein and demonstrated its ability to interact with Tanis by SPR analysis (FIG. 10D).

### EXAMPLE 4

### Tanis Protein Levels in the Sand Rat Tissues

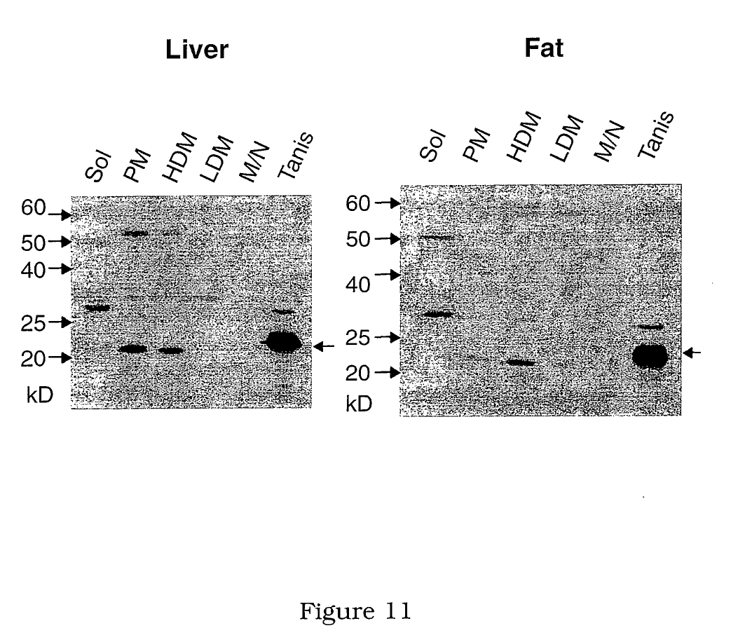

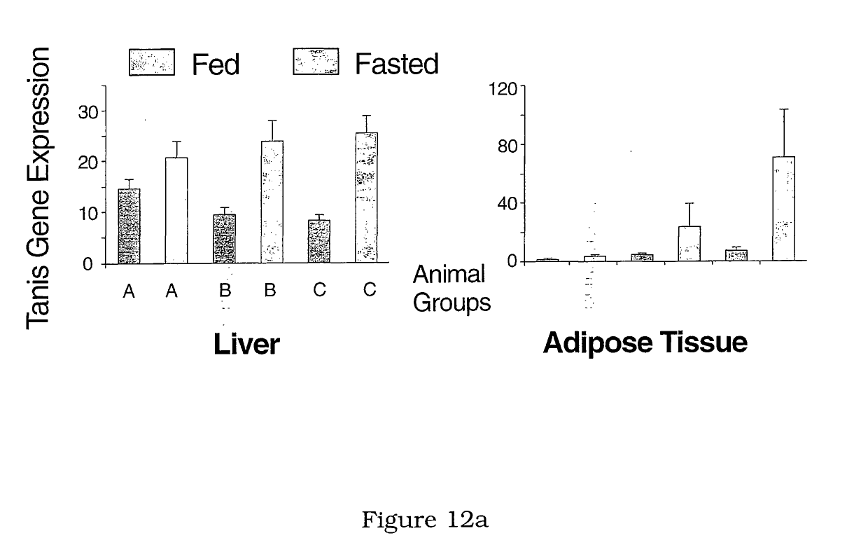

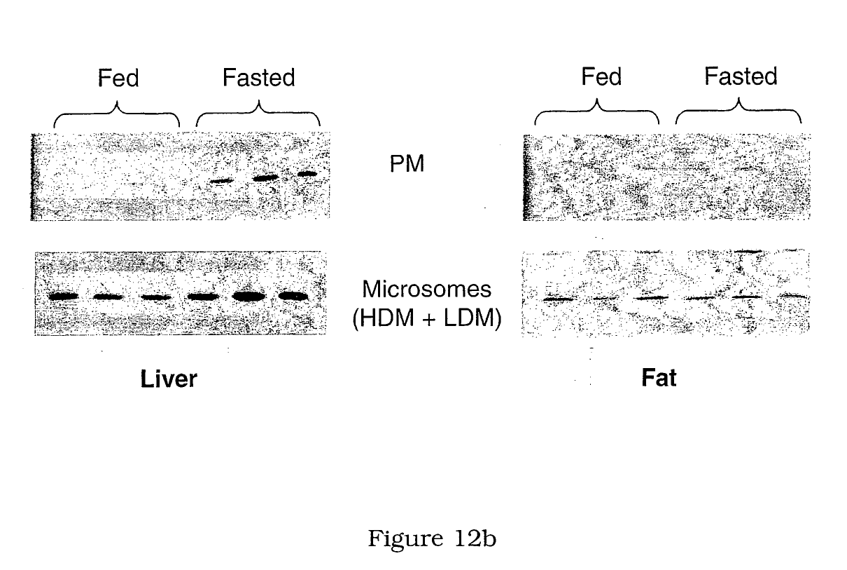

Tanis was discovered for its differential expression in diabetic/obese sand rats, but not in the healthy animals, during fasting in the liver and fat (FIG. 11A). To confirm and establish this differential expression on a protein level, plasma membrane and microsomes, which contained the Tanis protein, were isolated from the liver and fat of group C animals (diabetic and obese). Tanis protein levels in three fed and three fasted animals were compared on western blots using the anti-Tanis-C antibody. In the liver, fasting increased the protein level in the plasma membrane in particular and, to a lesser extent, in the microsomes (FIG. 12B). These data demonstrate that the Tanis protein is also responsive to fasting in the animals. However, little effect of fasting on Tanis protein level was observed in either the plasma membrane or the microsomes of in the fat tissue. Part of the reason for this discrepancy may be due to the high variability in its gene expression (FIG. 12A).

### EXAMPLE 5

### Tanis Expression in Response to Glucose in Hepatocytes

As reported earlier, Tanis gene expression was up-regulated by fasting in diabetic/obese, but not in healthy sand rats. One of the possible mechanisms is that the gene is regulated by glucose, since there is a significant decrease in plasma glucose concentration in the diabetic/obese animals but not in the healthy animals during a 24 h fasting.

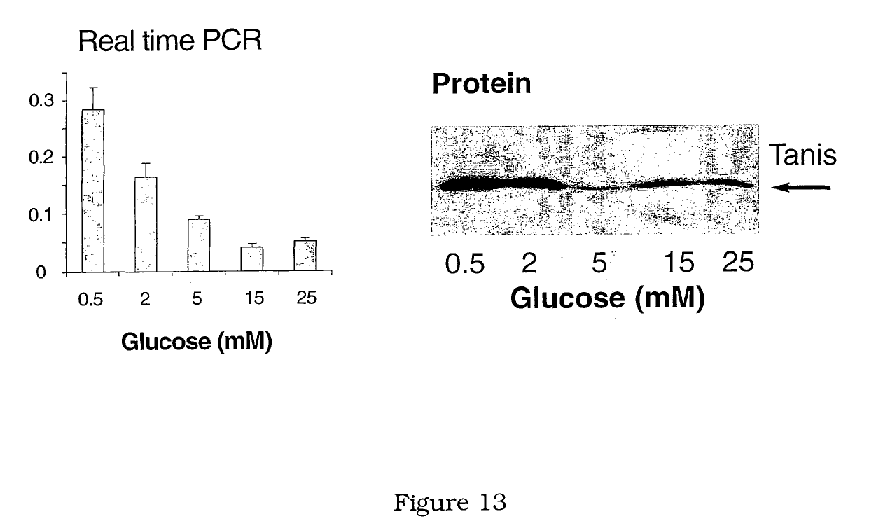

This hypothesis was examined in HepG2 hepatocytes. Tanis gene expression increased 5-6 fold when the cells were treated with low glucose, and there was a concomitantly increase in Tanis protein, as revealed by western blot (FIG. 13).

### EXAMPLE 6

### Glycogen Metabolism in H4IIE Cells

(i) Glycogen Content

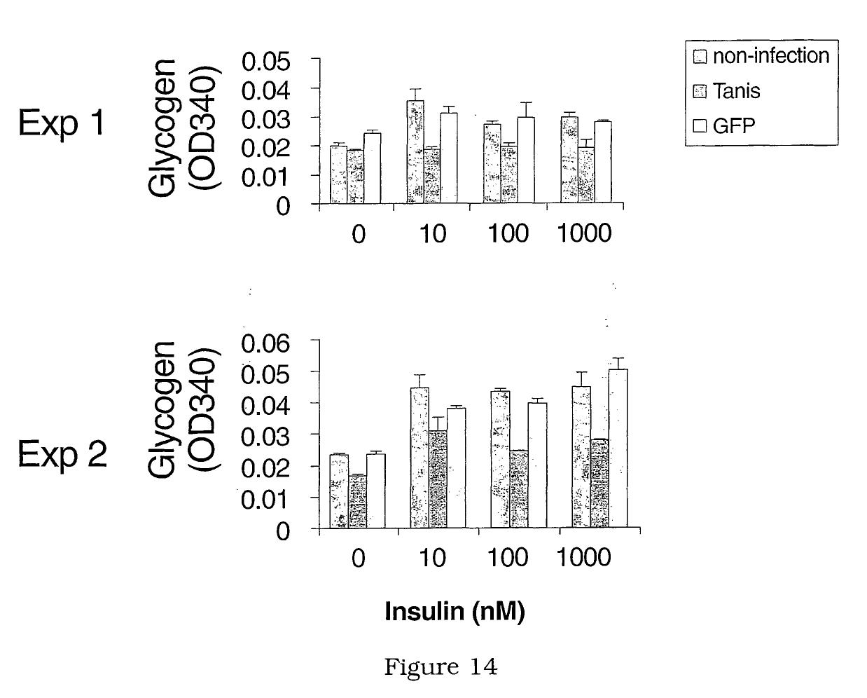

Glycogen content is the net results of its synthesis and breakdown, and is affected by a range of physiological stimuli or status. The fact that Tanis expression is enhanced during fasting in the liver cells indicates that it may be involved in glycogen metabolism. This hypothesis was addressed by measuring glycogen content and glycogen synthesis in H4IIE cells after Tanis overexpression using a recombinant adenovirus. Data from two separate experiments are presented in FIG. 14. Glycogen content in non-infected or GFP-infected cells increased with insulin treatment. However, cells infected with Tanis were less responsive to insulin. As a result, they contained significantly less glycogen after stimulation with insulin, suggesting that Tanis impairs the ability of insulin to stimulate glycogen synthesis.

(ii) Glycogen Synthesis

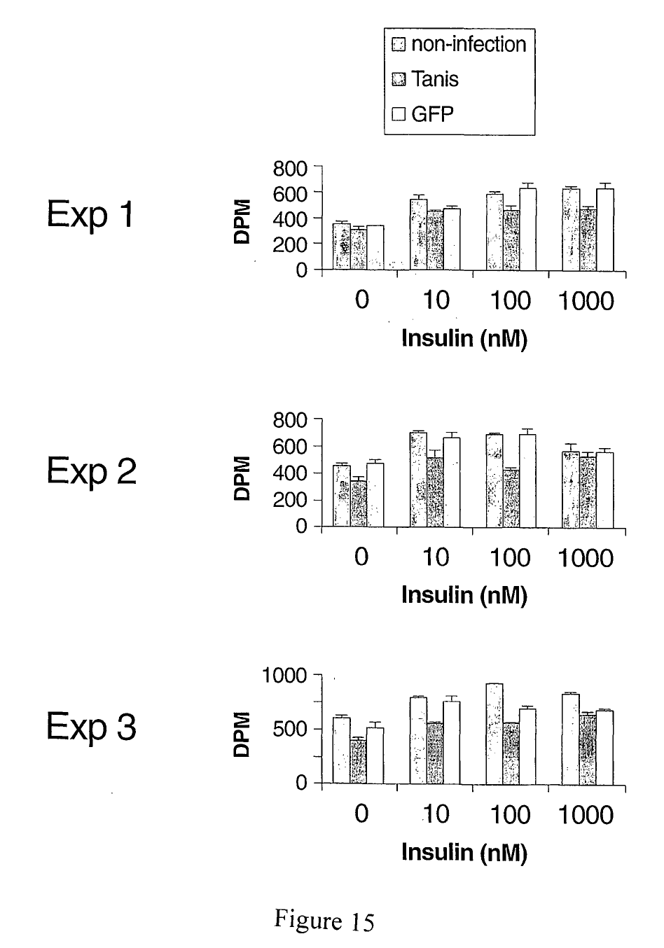

The glycogen synthesis experiment was repeated a number of times. Data from three independent experiments are presented here. In non-infected cells, glycogen synthesis increased about 50% by 100 nM insulin treatment. But higher insulin concentration (ie 1000 nM) did not produce a further increase. Infection with the GFP (control) virus had little effect on glycogen synthesis. In all three experiments, glycogen synthesis appeared to be decreased by Tanis overexpression (FIG. 15). These data are consistent with the glycogen content data, and suggest that the decrease in glycogen content is due to its impaired synthesis. Taking together, the data suggest that Tanis may have a role in regulation of glycogen synthesis in hepatocytes.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bausserman L L, Herbert P N, McAdam K P W J. (1980). J. Exp. Med. 152:641-656 Brandwein S R, Medsger T A Jr, Skinner M, Sipe J D, Rodnan G P, Cohen A S. (1984). Ann Rhem Dis 43(4):586-9
Brinckerhoff, C E. et al. (1989). Science 243(4891):655-7
Casl M T, Bulatovic G, Orlic P, Sabljar-Matovinovic M. (1995). Nephrol Dial Transplant 10(10):1901-4
Casl M T, Surina B, Glojnaric-Spasic I, Pape E, Jagarinec N, Kranjcevic S. (1995). Ann Clin Biochem 32(Pt 2):196-200
Chung T F, Sipe J D, McKee A, Finc R E, Schreiber B M, Liang J S, Johnson R J. (2000). Amyloid 7(2):105-10
Cunnane G, Grehan S, Geoghegan S, McCormack C, Shields D, Whitehead A S, Bresnihan B, Fitzgerald O. (2000). J Rheumatol 27(1):58-63
Cunnane G, Whitehead A S. (1999). Baillieres Best Pract Res Clin Rheumatol 13(4):615-28
Danesh J, Muir J, Wong Y K, Ward M, Gallimore J R, Pepys M B. (1999). Eur Heart J 20(13):954-9
Darling J C, Filteau S M, Kitundu J A, Kingamkono R R, Msengi A E, Tomkins A M. (1999). Acta Paediatr. 88(3):259-64
de Villiers W J, Varilek G W, de Beer F C, Guo J T, Kindy M S. (2000). Cytokine 12(9):1337-47, Diabetologia 41(10):1241-8
Erren M, Reinecke H, Junker R, Fobker M, Schulte H, Schurek J O, Kropf J, Kerber S, Breithardt G, Asmann G, Cullen P. (1999). Arterioscler Thromb Vase Biol. 19(10):2355-66
Falk R H, Comenzo R L, Skinner M. (1997). N Engl J Med 25 337(13):898-909
Grindulus et al. (1995). British Journal of Rheumatology 24:158-163
Hartmann A, Eide T C, Fauchald P, Bentdal O, Herbert J, Gallimore J R, Pepys M B. (1997). Nephrol Dial Transplant 12(1):161-6
Jensen L E, Whitehead A S. (1998). Biochem J 334(Pt 3):489-503
Kaneko K, Kakuta M, Ohtomo Y, Shimizu T, Yamashiro Y, Ogawa H, Manabe M. (2000). Dermatology 200(3):209-12
Kaysen G A. (1999). Miner Electrolyte Metab. 25(4-6):242-50
Kluve-Beckerman B, Malle E, Vitt H, Pfeiffer C, Benson M, Steinmetz A. (1991). Biochem. Biophys. Res. Commun. 181:1097-1102
Kluve-Beckerman B, Naylor S L, Marshall A, Gardner J C, Shows T B, Benson M D. (1986). Biochem. Ciophys. Res. Commun. 137:1196-1204
Lange U, Boss B, Teichmann J, Klor H, Neeck G. (2000). Rheumatol Int. 19(4):119-22
Liuzzo G, Biasucci L M, Gallimore J R, Grillo R L, Rebuzzi A G, Pepys M B, Mascri A. (1994). N. Engl. J. Med. 331(7):417
Meek R L, Urieli-Shovel S, Benditt E P. (1994). Proc. Natl. Acad. Sci. USA 91:3186
Mitchell T I, Coon C I, Brinckerhoff C E. (1991). J. Clin. Invest. 87(4):1177-85
Mozes G, Fredman N, Shainkin-Kestenbaum R. (1989). J Trauma 29(1):71-4
Muller T F, Trosch F, Ebel H, Grussner R W, Feiber H, Goke B, Greger B, Lange H. (1997). Transplant. 10(3):185-91
Pezzilli R, Melzi d'Eril G V, Morselli-Labate A M, Merlini G, Barakat B, Bosoni T. (2000). Dig Dis Sci. 45(6): 1072-8
Pickup J C, Crook M A., (1998)
Pickup J C, Mattock M B, Chusney G D, Burt D. (1997). Diabetologia 40(11):1286-92
Ray A and Ray B K. (1999). J. Immunol. 163:2143-2150
Ridker P M, Hennekens C H, Buring J E, Rifai N. (2000). N Engl J Med 23; 342(12):836-43
Ristori G, Laurenti F, Stacchini P, Gasperini C, Buttinelli C, Pozzilli C, Salvetti M. (1998). J Neuroimmunol 1; 88(1-2):9-12
Rohde L E, Arroyo L H, Rifai N, Creager M A, Libby P, Ridker P M, Lee R T. (1999). Arterioscler Thromb Vasc Biol 19(7):1695-9
Salazar A, Mana J, Fiol C, Hurtado I, Argimon J M, Pujol R, Pinto X. (2000). Atherosclerosis 152(2):497-502
Sellar G C, Jordon S A, Bickmore W A, Fantes J A, van Heyningen V, Whitehead A S. (1994). Genomics 19:221-227
Stefanadis C, Diamantopoulos L, Demellis J, Economou E, Tsiamis E, Toutouzas K, Vlachopoulos C, Toutouzas P. (2000). J Mol Cell Cardiol 32(1):43-52
Watson G, See C G, Woo P. (1994). Genomics 23:694-696
Whicher J T, Chambers R E, Higginson J, Nashef L, Higgins PG. (1985). J Clin Pathol 38(3):312-6
Yang F, de Villiers W J, Lee E Y, McClain C J, Varilek G W. (1999). J Lab Clin Med., 134(4):378-85

1. A method of modulating the functional activity of an apolipoprotein or derivative, homologue, analogue, chemical equivalent or mimetic thereof in a subject, said method comprising administering to said subject an effective amount of an agent for a time and under conditions sufficient to modulate the interaction of Tanis or derivative, homologue, analogue, chemical equivalent or mimetic thereof with said apolipoprotein.

2. The method according to claim 1 wherein upregulating said interaction upregulates said apolipoprotein functional activity and downregulating said interaction down-regulates said apolipoprotein functional activity.

3. The method according to claim 1 or 2 wherein said apolipoprotein is serum amyloid A.

4. A method of modulating the functional activity of Tanis or derivative, homologue, analogue, chemical equivalent or mimetic thereof in a subject, said method comprising administering to said subject an effective amount of an agent for a time and under conditions sufficient to modulate the interaction of an apolipoprotein or derivative, homologue, analogue, chemical equivalent or mimetic thereof with said Tanis.

5. The method according to claim 4 wherein upregulating said interaction upregulates said Tanis functional activity and downregulating said interaction downregulates said Tanis functional activity.

6. The method according to claim 4 or 5 wherein said apolipoprotein is serum amyloid A.

7. A method for the treatment and/or prophylaxis of a condition characterised by aberrant, unwanted or otherwise inappropriate apolipoprotein mediated functional activity in a mammal, said method comprising administering to said mammal an effective amount of an agent for a time and under conditions sufficient to modulate the interaction of Tanis with said apolipoprotein.

8. A method for the treatment and/or prophylaxis of a condition characterised by aberrant, unwanted or otherwise inappropriate Tanis mediated functional activity in a mammal, said method comprising administering to said mammal an effective amount of an agent for a time and under conditions sufficient to modulate the interaction of apolipoprotein with said Tanis.

9. The method according to claim 7 or 8 wherein upregulating said interaction upregulates said apolipoprotein or Tanis mediated functional activity and down-regulating said interaction downregulates said apolipoprotein or Tanis functional activity.

10. The method according to claim 7-9 wherein said apolipoprotein is serum amyloid A.

11. The method according to any one of claims 7-10 wherein said condition is type II diabetes, inflammation, cardiovascular disease, transplantation rejection, infection, sarcoidosis, Alzheimer's disease, nephropathy, abdominal aortic aneurism or obesity.

12. Use of an agent in the manufacture of a medicament for the treatment of a condition in a mammal, which condition is characterised by aberrant, unwanted or otherwise inappropriate apolipoprotein mediated functional activity, wherein said agent modulates the interaction of Tanis with an apolipoprotein.

13. Use according to claim 12 wherein upregulating said interaction upregulates said apolipoprotein functional activity and downregulating said interaction downregulates said apolipoprotein functional activity.

14. Use according to claim 12 or 13 wherein said apolipoprotein is serum amyloid A.

15. Use of an agent in the manufacture of a medicament for the treatment of a condition in a mammal, which condition is characterised by aberrant, unwanted or otherwise inappropriate Tanis mediated functional activity, wherein said agent modulates the interaction of Tanis with an apolipoprotein.

16. Use according to claim 15 wherein upregulating said interaction upregulates said Tanis functional activity and downregulating said interaction downregulates said Tanis functional activity.

17. Use according to claim 15 or 16 wherein said apolipoprotein is serum amyloid A.

18. A pharmaceutical composition comprising an agent together with one or more pharmaceutically acceptable carrier and/or diluents, wherein said agent modulates the interaction of Tanis or derivative, homologue, analogue, chemical equivalent or mimetic thereof with an apolipoprotein or derivative, homologue, analogue, chemical equivalent or mimetic thereof, which modulation regulates Tanis and/or apolipoprotein functional activity.

19. An agent, which agent modulates the interaction of Tanis or derivative, homologue, analogue, chemical equivalent or mimetic thereof with an apolipoprotein or derivative, homologue, analogue, chemical equivalent or mimetic thereof, when used in accordance with the method of any one of claims 1-11.

20. A method for detecting an agent capable of modulating the interaction of Tanis with an apolipoprotein or its derivative, homologue, analogue, chemical equivalent or mimetic thereof, said method comprising contacting an in vitro system containing said Tanis and said apolipoprotein with a putative agent and detecting an altered expression phenotype associated with said interaction.

21. The agent identified according to the method of claim 20.
